(19) United States
(12) Patent Application Publication
(10) Pub. No.: US 2003/0157519 A1
(43) Pub. Date: Aug. 21, 2003

(54) Recombinant renilla reniformis system for bioluminescence resonance energy transfer

(75) Inventors: Vivan Q. Zhang, San Diego, CA (US); Peter E. Vaillancourt, Del Mar, CA (US)

Correspondence Address: PALMER & DODGE, LLP, KATHLEEN M. WILLIAMS / STR, 111 HUNTINGTON AVENUE, BOSTON, MA, 02199, US

(73) Assignee: Stratagene

(21) Appl. No.: 10/265,031

(22) Filed: Oct. 4, 2002

Related U.S. Application Data
(60) Provisional application No. 60/329,354, filed on Oct. 15, 2001.

(51) Int. Cl. C12Q 1/68; G01N 33/53; G01N 33/537; G01N 33/543; C12Q 1/66; C12N 9/06; C07H 21/04; C12P 21/02; C12N 5/06

(52) U.S. Cl. 435/6; 435/8; 435/7.92; 435/69.1; 435/191; 435/320.1; 435/325; 536/23.2

(57) ABSTRACT

The invention relates to compositions comprising a first fusion protein comprising a first polypeptide domain and a R. reniformis luciferase and a second fusion protein comprising a second polypeptide domain and a R. reniformis GFP. The invention also relates to compositions comprising one or more polynucleotides encoding a first fusion protein comprising a first polypeptide domain and a R. reniformis luciferase and a second fusion protein comprising a second polypeptide domain and a R. reniformis GFP. The invention also relates to methods and kits for detecting protein-protein interactions, determining the location of a protein-protein interaction, identifying cells wherein there is a protein-protein interaction of interest, and screening for a candidate modulator that increases or decreases the amount of a protein-protein interaction.

### RELATED APPLICATION(S)

This application claims the benefit of U.S. Provisional Application No. 60/329354, filed on Oct. 15, 2001. The entire teachings of the above application is incorporated herein by reference.

### FIELD OF THE INVENTION

The invention relates to BRET-based assays.

### BACKGROUND OF THE INVENTION

Resonance energy transfer between two chromophores is a quantum mechanical process that is exquisitely sensitive to the distance between the donor and acceptor chromophores and their relative orientation in space (Wu & Brand (1994) Anal. Biochem. 218 1-13). Efficiency of energy transfer is inversely proportional to the 10⁶ power of chromophore separation. In practice, the useful distance range is about 10 to 100 Angstroms, which has made resonance energy transfer a very useful technique for studying the interactions of biological macromolecules. A variety of fluorescence-based FRET biosensors have been constructed, initially employing chemical fluors conjugated to proteins or membrane components, and more recently, using pairs of spectrally distinct GFP mutants (Giuliano & Taylor (1998) Trends Biotech. 16: 99-146; Tsien (1998) Annu. Rev. Biochem. 67:509-44).

Bioluminescence Resonance Energy Transfer (BRET) is a natural resonance energy transfer phenomenon that was first inferred from studies of the hydrozoan Obelia (Morin & Hastings (1971) J. Cell Physiol. 77:313-18), whereby the green bioluminescent emission observed in vivo was shown to be the result of energy transfer from the luciferase to an accessory green fluorescent protein (GFP). BRET was soon thereafter observed in the hydrozoan Aequorea victoria and the anthozoan Renilla reniformis. Although energy transfer in vitro between purified luciferase and GFP has been demonstrated in Aequorea (Morise et al. (1974) Biochemistry 13: 2656-62) and Renilla (Ward & Cormier (1976) J. Phys. Chem. 80:2289-91) systems, a key difference is that in solution, efficient radiationless energy transfer occurs only in Renilla, apparently due to the pre-association of one luciferase molecule with one GFP homodimer (Ward & Cormier (1978) Photochem. Photobiol. 27:389-96). The blue (486 nm) luminescent emission of Renilla luciferase can be completely converted to a narrow band green emission (508 nm) upon addition of proper amounts of Renilla GFP (Ward & Cormier (1976) J. Phys. Chem. 80: 2289-91). By virtue of the non-radiative energy transfer, the quantum yield of the luciferase is increased. The strict dependence of BRET on the close proximity between the energy donors and acceptors makes this an efficient system for monitoring protein-protein interactions in living cells.

BRET has recently become a popular readout for cell-based assays for high-throughput screening (HTS) due to the ability of cells to take up the luciferase substrate without the requirement for cell lysis or manipulation (Xu et al., 1999 Proc Natl Acad Sci USA 96:151-6; Angers et al., 2000, Proc. Natl. Acad. Sci USA, 97:3684-9; McVey et al., 2001, J. Biol. Chem., 276:14092-9; Kroeger et al., 2001, 276:12736-43). However, these assays use the R. reniformis luciferase coupled with the Aequorea GFP variant EYFP or with the wild-type aequorea GFP and a synthetic substrate (“Deep BlueC”). The major drawback to the use of these assay conditions is that there is minimal separation of the emission spectra from luciferase and GFP, resulting in a poor signal to noise ratio. This system also suffers from the very poor quantum yield for the luciferase substrate DeepBlueC.

Developing technologies such as high throughput screening for candidate drugs (using high throughput screening (HTS) protocols), biochips and environmental monitoring systems would benefit greatly from modular biosensors where the signal of a rare target “hit” (e.g., complex formation between two polypeptides) is unambiguously (statistically) distinguishable from the huge excess of “non-hits”). Current genetically encoded FRET and bioluminescence-based biosensors display hit signals that very often are less than two-fold greater than non-hit signals, and are at best a few-fold greater (Xu et al. (1999) Proc. Natl. Acad. Sci USA 96: 151-156; Miyawaki et al. (1997) Nature 388:882-7).

In an effort to improve the sensitivity of detection of protein-protein interactions, research has focused on BRET assays in which both luciferase and GFP originate from the same species. In this regard, U.S. Pat. No. 6,232,107 discloses a BRET system that includes a bioluminescence generating system comprising Renilla mulleri luciferase and Renilla reniformis, kollokeri or Renilla mulleri GFP. There is therefore a need in the art for a BRET assay system with increased sensitivity and a decreased signal to noise ratio.

All references cited herein, including published patent applications and publications, are incorporated by reference in their entirety.

### SUMMARY OF THE INVENTION

The invention provides for a composition comprising a first polynucleotide comprising an expression cassette containing a sequence encoding R. reniformis luciferase and a second polynucleotide comprising an expression cassette containing a sequence encoding R. reniformis GFP.

In one embodiment, the polynucleotide sequence of R. reniformis GFP is humanized.

In another embodiment, the polynucleotide sequence of R. reniformis GFP comprises the sequence of SEQ ID NO: 1.

In another embodiment, the composition further comprises a substrate for luciferase.

In another embodiment, the substrate is coelentrazine.

In one embodiment, the fused heterologous polypeptide domain is fused to the amino-terminal end of the R. reniformis GFP or variant thereof, wherein R. reniformis GFP is encoded by a humanized GFP polynucleotide sequence.

In one embodiment, the fused heterologous polypeptide domain is fused to the amino-terminal end of the R. reniformis luciferase or variant thereof.

In another embodiment, the fused heterologous polypeptide domain is fused to the carboxy-terminal end of the R. reniformis GFP or variant thereof, wherein R. reniformis GFP is encoded by a humanized GFP polynucleotide sequence.

In another embodiment, the fused heterologous polypeptide domain is fused to the carboxy-terminal end of the R. reniformis luciferase or variant thereof.

In another embodiment, the fused heterologous polypeptide domain is fused to the R. reniformis GFP or variant thereof via a linker sequence, wherein R. reniformis GFP is encoded by a humanized GFP polynucleotide sequence.

In another embodiment, the fused heterologous polypeptide domain is fused to the R. reniformis Luciferase or variant thereof via a linker sequence.

The invention also provides for a method of detecting protein: protein interactions comprising the steps of providing a first fusion protein comprising a first polypeptide domain fused to a R. reniformis luciferase polypeptide, and a second fusion protein comprising a second polypeptide domain fused to a R. reniformis GFP polypeptide and a substrate for luciferase; mixing the first and second fusion polypeptides and said substrate; and detecting BRET induced fluorescent emission from said R. reniformis GFP, wherein the fluorescent emission from the R. reniformis GFP indicates protein:protein interaction between the first and second polypeptide domains.

In one embodiment, the R. reniformis GFP polypeptide is encoded by a humanized polynucleotide sequence.

In another embodiment, the R. reniformis GFP polypeptide is encoded by a polynucleotide sequence comprising SEQ ID NO: 1.

In another embodiment, the substrate is coelentrazine.

In another embodiment, the method is performed in a cell.

In another embodiment, the method is performed in cell membranes comprising the fusion polypeptides.

The invention also provides for a method of determining the location of a protein:protein interaction between two polypeptide domains, said method comprising the steps of: a) providing a first fusion polynucleotide sequence encoding a first fusion polypeptide comprising a first polypeptide domain and a R. reniformis GFP polypeptide, and providing a second fusion polynucleotide sequence encoding a second fusion polypeptide comprising a second polypeptide domain and a R. reniformis luciferase polypeptide; b) introducing the first and second fusion polynucleotide sequences to a cell; c) adding a substrate for luciferase to the cells; and d) determining the cellular location of a fluorescent emission from said R. reniformis GFP, wherein the fluorescent emission from the R. reniformis GFP indicates the cellular location of the protein:protein interaction between the first and the second polypeptide domains.

Preferably, the fluorescent emission is BRET-induced fluorescent emission.

In one embodiment, the R. reniformis GFP polypeptide is encoded by a humanized polynucleotide sequence.

In another embodiment, the R. reniformis GFP polypeptide is encoded by a polynucleotide sequence comprising SEQ ID NO: 1.

In another embodiment, the substrate is coelentrazine.

The invention also provides for a method of identifying cells in which there is a protein:protein interaction between two polypeptide domains of interest, comprising the steps of: a) introducing a polynucleotide sequence encoding a first fusion polypeptide comprising a first polypeptide domain and R. reniformis GFP, and a second polynucleotide sequence encoding a second fusion polypeptide comprising a second polypeptide domain and R. reniformis luciferase into a population of cells; b) adding a substrate for luciferase to the cells; and c) detecting fluorescent emission from said R. reniformis GFP, wherein said fluorescent emission from said R. reniformis GFP identifies a cell in which a protein:protein interaction between said first and said second polypeptide domains has occurred.

Preferably, the fluorescent emission is BRET-induced fluorescent emission.

In one embodiment, the R. reniformis GFP polypeptide is encoded by a humanized polynucleotide sequence.

In another embodiment, the R. reniformis GFP polypeptide is encoded by a polynucleotide sequence comprising SEQ ID NO: 1.

In another embodiment, the substrate is coelentrazine.

In another embodiment, detection involves fluorescent activated cell sorter (FACS) analysis.

In another embodiment, the method is performed in tissues obtained from a transgenic animal.

In another embodiment, the population of cells are transformed with a single polynucleotide sequence encoding both a first fusion protein comprising a first polypeptide domain and a R. reniformis GFP polypeptide, and a second fusion protein comprising a second polypeptide domain and a R. reniformis luciferase polypeptide.

The invention also provides for a method of screening for a candidate modulator that increases or decreases a protein:protein interaction between two polypeptide domains, comprising the steps of: a) providing a first fusion protein comprising a first polypeptide domain and R. reniformis GFP polypeptide, and a second fusion protein comprising a second polypeptide domain and R. reniformis luciferase polypeptide; b) mixing the first and second fusion polypeptides with the candidate modulator under conditions that permit binding of the fusion polypeptides to each other; c) adding a substrate for luciferase; d) measuring fluorescent emission from the R. reniformis GFP, wherein the fluorescent emission from the R. reniformis GFP indicates a protein:protein interaction between the first and second polypeptide domains; and e) comparing the amount of R. reniformis GFP fluorescence in the presence and absence of the candidate modulator.

Preferably, the fluorescent emission is BRET-induced fluorescent emission.

In one embodiment, R. reniformis GFP polypeptide is encoded by a humanized polynucleotide sequence.

In another embodiment, the R. reniformis GFP polypeptide is encoded by a polynucleotide comprising the sequence of SEQ ID NO: 1.

In another embodiment, the substrate is coelentrazine.

In another embodiment, the method is performed in a living cell.

In another embodiment, the first and second fusion polypeptides are present in a cell membrane.

In another embodiment, the candidate modulator is selected from the group consisting of a natural or synthetic peptide, a polypeptide, an antibody or antigen-binding fragment thereof, a lipid, a carbohydrate, a nucleic acid, and a small organic molecule.

In another embodiment, the step of measuring comprises detecting a change in the level of fluorescent emission from said R. reniformis GFP in the presence of a candidate modulator as compared to the absence of a candidate modulator.

In another embodiment, the method is performed in a microarray.

In another embodiment, the first polypeptide domain and said second polypeptide domain are identical.

In another embodiment, the first and second polypeptide domains are receptor domains or portions thereof.

The invention also provides for a kit for detecting a protein:protein interaction, determining the cellular location of a protein:protein interaction in a cell, identifying cells in which there is a protein:protein interaction, or screening for agents that modulate a protein:protein interaction, comprising a first recombinant expression vector encoding a R. reniformis luciferase polypeptide and a second recombinant expression vector encoding a R. reniformis luciferase polypeptide and packaging materials therefore.

In one embodiment, the kit comprises a single recombinant expression vector encoding a R. reniformis luciferase polypeptide and a R. reniformis GFP polypeptide and packaging materials therefore.

In another embodiment, the R. reniformis GFP polypeptide is encoded by a humanized polynucleotide sequence.

In another embodiment, the R. reniformis GFP polypeptide is encoded by a polynucleotide sequence comprising SEQ ID NO: 1.

In another embodiment, the kit further comprises a substrate for luciferase.

In another embodiment, the substrate is coelentrazine.

As used herein, “BRET” or bioluminescence resonance energy transfer” refers to non-radiative luciferase-to-fluorescent protein (FP) energy transfer. It differs from (Fluorescence Resonance Energy Transfer), which historically has been used for energy transfer between chemical fluors, but more recently has been applied to energy transfer between Aequorea GFP spectral variants.

As used herein, a “BRET” system refers to the combination of a FP and luciferase for resonance energy transfer and BRET refers to any method in which the luciferase is used to generate light energy upon reaction with a luciferin, which is then non-radiatively transferred to a FP. The transferred energy, particularly to a GFP, is focused, shifted and emitted at a different wavelength. The “BRET system” also may include a bioluminescence generating system (e.g. an FP). Alterations in the proximity of the FP and GFP will be reflected in changes in the emission spectra of light produced by the luciferase. As a result, the pair can function as a sensor of external events.

As used herein, a “biosensor” (or sensor) refers to a BRET system for use to detect alterations in the environment in vitro or in vivo in which the BRET system is used.

As used herein, a “polypeptide” refers to a protein having biological activity and which may have a binding activity. A “polypeptide” also refers to a “binding” domain of a protein which is a region of the protein that binds to a cognate polypeptide or domain thereof. Binding of two cognate polypeptides normally produces a biological effect.

As used herein, “non-radiative” refers to the transfer of energy from an energy donor to an energy acceptor without the emission of light by the donor.

As used herein, “protein-protein interaction” or “binding” refers to the specific complimentary recognition and association of two proteins with a dissociation constant, Kd of preferably 10⁻⁵M, more preferably 10⁻⁷M, most preferably 10⁻⁹M or less.

As used herein, the term “fusion polypeptide” or “fusion protein” refers to a polypeptide that is comprised of two or more amino acid sequences, wherein each amino acid sequence encodes a protein or a portion thereof, wherein the two or more amino acid sequences are not found linked in nature, and wherein the two or more amino acid sequences are physically linked by a peptide bond.

As used herein, “domain” refers to a region of a protein that is at least 2 amino acids less than the whole protein and which retains at least the biological activity of the whole protein. A domain may range in size from 10-1000 amino acids in length, e.g. 50-60 amino acids, 100-400 amino acids or 200-300 amino acids. A “domain” refers to a functional unit of a complete protein having a biological activity of the complete protein. For example, a “domain” of a protein useful according to the invention may refer to a region of a protein that binds to a second protein.

As used herein, “cellular compartment” refers to organelles (nucleus, mitochondria, endoplasmic reticulum, Golgi etc), a membrane, cell envelope or cell wall, or a preparation of any thereof.

As used herein, “GFP” refers to Green Fluorescent Proteins, a class of intrinsically fluorescent chromoproteins that are isolated from certain bioluminescent coelenterates. For example, the Renilla reniformis GFP, when excited at ˜500 nm, gives a fluorescence emission, as defined herein, with a peak at 508-510 nm. The invention also contemplates a humanized version of R. reniformis GFP.

As used herein, “humanized GFP” refers to a Renilla reniformis GFP polynucleotide coding sequence in which one or more, (for example, 2, 3, 4, 5, 10, 20, 50, 75, 100, 200, 500 or more, including, in certain embodiments, all the codons of the polynucleotide coding sequence for the non-human GFP polypeptide (i.e., a polypeptide not naturally expressed in humans) have been altered to a codon sequence more preferred for expression in human cells.

A polypeptide coding sequence is herein referred to as “humanized” if one or more codons is altered from the natural coding sequence to a codon which is utilized in a human but not in Renilla. Because there are 64 possible combinations of the 4 DNA nucleotides in codon groups of 3, the genetic code is redundant for many of the 20 amino acids. Each of the different codons for a given amino acid encodes the incorporation of that amino acid into a polypeptide. However, within a given species there tends to be a preference for certain of the redundant codons to encode a given amino acid. The “codon preference” of R. reniformis is different from that of humans (this codon preference is usually based upon differences in the level of expression of the tRNAs containing the corresponding anticodon sequences). In order to obtain high expression of a non-human gene product in human cells, it is advantageous to change one or more non-preferred codons to a codon sequence that is preferred in human cells. Table 1 shows the preferred codons for human gene expression. A codon sequence is preferred for human expression if it occurs to the left of a given codon sequence in the table. Optimally, but not necessarily, less preferred codons in a non-human polynucleotide coding sequence are humanized by altering them to the codon most preferred for that amino acid in human gene expression. As used herein, a GFP is “humanized” if the amount of fluorescent polypeptide expressed in a human cell from a “humanized GFP” polynucleotide sequence is at least two-fold greater, on either a mass or a fluorescence intensity scale per cell, than the amount expressed from an equal amount or number of copies of, a non-humanized GFP polynucleotide.

As used herein, “luciferase” refers to an oxygenase that catalyzes a light emitting reaction. Thus, luciferase refers to an enzyme or photoprotein that catalyzes a bioluminescent reaction. The luciferases, such as firefly and Gaussia and Renilla luciferases are enzymes, which act catalytically and are unchanged during the bioluminescence generating reaction. The vector pRL-CMV (GenBank accession number: AF025843) contains a wild type version of R. reniformis luciferase that is also commercially available from Promega. The invention also contemplates a humanized version of R. reniformis luciferase. The vector phRL-CMV (GenBank accession number: AF362549) contains a humanized version of R. reniformis luciferase that is also commercially available from Promega.

As used herein, a “bioluminescent reaction” is a reaction that produces bioluminescence.

As used herein, “substrate for luciferase” refers to a compound that is oxidized in the presence of a luciferase, as well as any necessary activators, and generates light. A “substrate for luciferase”, according to the invention, includes luciferin but can be any substrate that undergoes oxidation in a bioluminescence reaction. Bioluminescent substrates, according to the invention, includes any luciferin or analog thereof or any synthetic compound with which a luciferase interacts to generate light. Preferred substrates are those that are oxidized in the presence of a luciferase. Bioluminescent substrates, thus, include those compounds that those of skill in the art recognize as luciferins. Luciferins, for example, include firefly luciferin, Cypridina [also known as Vargula] luciferin [coelenterazine], bacterial luciferin, as well as synthetic analogs of these substrates or other compounds that are oxidized in the presence of a luciferase in a reaction the produces bioluminescence.

A “cell”, useful according to the invention, can be any eukaryotic or prokaryotic cell.

As used herein, “fluorescent emission” refers to the light emitted from a fluorescent protein. “Fluorescent emission” can be detected in a bioluminescent resonance energy transfer assay” as described herein. Fluorescent emission can be detected by the methods of fluorescence activated cell sorting (FACS) or fluorescence microscopy, also as described herein.

As used herein, “cell membrane” preparation refers to a preparation of cellular lipid membranes. As used herein, a “cell membrane” preparation is distinct from a cellular homogenate, in that at least a portion (i.e., at least 10%, and preferably more) of non-membrane-associated cellular constituents has been removed from the homogenate. “Membrane associated” refers to a polypeptide that is either integrated into a lipid membrane or is physically associated with a component that is integrated into a lipid membrane.

As used herein, “introducing” refers to the delivery of a nucleic acid construct into a cell using standard methods such as transfection, injection or electroporation.

As used herein, “determining the location” refers to using fluorescence microscopy to detect the cellular location of BRET-induced fluorescence emitted by GFP fusion proteins. A location can be any intracellular, including a location in an organelle of the cell or an extracellular location, for example, a location in the cell membrane.

As used herein, “recombinant vector” refers to a discrete genetic element that is used to introduce heterologous nucleic acid into cells for either expression or replication thereof. Selection and use of such vehicles are well within the skill of the artisan. A recombinant expression vector includes vectors capable of expressing nucleic acids that are operatively linked to regulatory sequences, such as promoter regions that are capable of effecting expression of such nucleic acids. Thus, an expression vector refers to a recombinant DNA or RNA construct, such as a plasmid, a phage, recombinant virus or other vector that, upon introduction into an appropriate host cell, results in expression of the cloned nucleic acid. Appropriate expression vectors are well known to those of skill in the art and include those that are replicable in eukaryotic cells and/or prokaryotic cells including those that remain episomal or those, which integrate into the host cell genome.

As used herein, “polynucleotide” refers to a covalently linked sequence of nucleotide bases (i.e., deoxyribonucleotides for DNA) in which the 3′ position of the pentose of one nucleotide is joined by a phosphodiester group to the 5′ position of the pentose of the next nucleotide. The term “polynucleotide”, as used herein, is interchangeable with the term “nucleic acid”.

As used herein, a “receptor” refers to a molecule, such as a protein, glycoprotein and the like, that can specifically (non-randomly) bind to another molecule, for example an extracellular signaling molecule, and thereby initiates a response in a cell.

As used herein, “detecting”, refers to the use of a plate reader or similar apparatus that is capable of detecting and measuring BRET-induced fluorescence.

As used herein, “transgenic animal” refers to any animal, preferably a non-human mammal, bird, fish or an amphibian, in which one or more of the cells of the animal contain heterologous nucleic acid introduced by way of intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extra-chromosomally replicating DNA. In the typical transgenic animals described herein, the transgene causes cells to express a first fusion polypeptide comprising a first polypeptide domain and a R. reniformis humanized GFP-derived polypeptide, and a second fusion polypeptide comprising a second polypeptide domain and a R. reniformis luciferase(luc)-derived polypeptide. However, transgenic animals in which the recombinant gene is silent are also contemplated, as for example, the FLP or CRE recombinase dependent constructs as described herein. Moreover, “transgenic animal” also includes those recombinant animals in which gene disruption of one or more genes is caused by human intervention, including both recombination and antisense techniques.

As used herein, “tissue” refers to an aggregate of cells that performs a particular function in an organism. As used herein, “tissue” as used herein refers to cellular material from a particular physiological region. The cells in a particular tissue can comprise several different cell types. A non-limiting example of this would be brain tissue that further comprises neurons and glial cells, as well as capillary endothelial cells and blood cells, all contained in a given tissue section or sample. In addition to solid tissues, “tissue” is also intended to encompass non-solid tissues, such as blood.

As used herein, “fluorescence activated cell sorting (FACS analysis)” refers to the method of sorting cells wherein cells are stained with or express one or more fluorescent markers. In this method, cells are passed through an apparatus that excites and detects fluorescence from the marker(s). Upon detection of fluorescence in a given portion of the spectrum by a cell, the FACS apparatus allows the separation of that cell from those cells not expressing that fluorescence spectrum.

As used herein, a polylinker or “multiple cloning site” refers to sites within a vector that permit insertion and cloning of DNA fragments. A sequence of nucleotides adapted for directional ligation, i.e. a polylinker, is a region of the DNA expression vector that (1) operatively links for replication and transport the upstream and downstream translatable DNA sequences, and (2) provides a site for directional ligation of a DNA sequence into the vector. Typically, a directional polylinker is a sequence of nucleotides that defines two or more restriction endonuclease recognition sequences. Upon restriction cleavage, the two sites yield cohesive termini to which a translatable DNA sequence can be ligated to the DNA expression vector. Preferably, the two restriction sites provide, upon restriction cleavage, cohesive termini that are non-complementary and thereby permit directional insertion of a translatable DNA sequence into the cassette. Where the sequence of nucleotides adapted for directional ligation defines numerous restriction sites, it is referred to as a “multiple cloning site”.

As used herein, “population of cells” refers to a plurality of cells, preferably, but not necessarily of the same type or strain.

As used herein, “plurality” refers to more than two. Plurality, according to the invention, can be 3 or more, 100 or more, or 1000 or more.

As used herein, a “modulator” refers to any compound that increases the fluorescent emission from R. reniformis humanized GFP-derived polypeptide, wherein said fluorescent emission from said R. reniformis humanized GFP polypeptide indicates an increased or decreased protein:protein interaction between two polypeptide domains, by at least 2-fold, preferably 5-fold, more preferably 10-fold and most preferably, 100-fold or more (i.e., 150-fold, 200-fold, 250-fold, 500-fold, 1000-fold, 10,000-fold, etc . . . ), as compared to the fluorescent emission in the absence of a modulator according to the invention. A modulator also refers to a compound that is capable of increasing or decreasing the fluorescent emission by at least 10%, preferably 15-25%, more preferably 25-50% and most preferably 50-100%, as compared to fluorescent emission in the absence of a compound. A “modulator” includes an agonist, an antagonist, an inverse agonist, a protease inhibitor, a protease activator, or any compound that increases or decreases the fluorescent emission from a R. reniformis humanized GFP-derived polypeptide. A modulator can be a protein, a nucleic acid, an antibody or fragment thereof, a peptide, etc . . . . Candidate modulators can be natural or synthetic compounds, including, for example, small molecules, compounds contained in extracts of animal, plant, bacterial or fungal cells, as well as conditioned medium from such cells.

As used herein, “measuring” refers to using a fluorometer or comparable equipment to detect or quantitate BRET-induced fluorescence for instance in a 96 well format.

As used herein, “comparing” refers to analyzing or evaluating the difference in the amount of R. reniformis GFP fluorescence in the presence and absence of a candidate modulator.

As used herein, “change in the level of fluorescent emission” refers to an increase or decrease in the amount of fluorescence emitted by an R. reniformis GFP polypeptide fusion in a BRET system of the invention as compared to a standard in a given assay, or as compared to the amount of fluorescence emitted by a R. reniformis GFP in the presence versus the absence of a candidate modulator. A “change in the level of fluorescence emission” is preferably at least 10%, more preferably at least 50 or most preferably greater than 100%.

As used herein, a “standard BRET system” refers to a control BRET assay using a cell line expressing both GFP and luciferase fusion proteins in which the protein domain moieties are known to interact specifically.

As used herein, “microarray”, refers to a plurality of unique biomolecules attached to one surface of a solid support. Preferably, a biomolecule of the invention is a fusion protein, as described herein. In this embodiment, the microarray of the invention comprises fusion protein molecules that are immobilised on a solid support at a density exceeding 20 different biomolecules/cm² wherein each of the biomolecules is attached to the surface of the solid support in a non-identical pre-selected region. Suitable solid supports are available commercially, and will be apparent to the skilled person. The supports may be manufactured from materials such as glass, ceramics, silica and silicon. The supports usually comprise a flat (planar) surface, or at least an array in which the molecules to be interrogated are in the same plane. In one embodiment, the array comprises at least 500 different biomolecules attached to one surface of the solid support. In another embodiment, the array comprises at least 10 different biomolecules attached to one surface of the solid support. In yet another embodiment, the array comprises at least 10,000 different biomolecules attached to one surface of the solid support.

As used herein, “antibody” refers to a conventional immunoglobulin molecule, as well as fragments thereof which are also specifically reactive with one of the subject polypeptides or fusion proteins. Antibodies can be fragmented using conventional techniques and the fragments screened for utility. For example, F(ab)2 fragments can be generated by treating an antibody with pepsin. The resulting F(ab)2 fragment can be treated to reduce disulfide bridges to produce Fab fragments. An antibody of the present invention is further intended to include bispecific, single-chain, and chimeric and humanized molecules having affinity for a polypeptide conferred by at least one CDR region of the antibody. The antibodies may be monoclonal or polyclonal and may include a hypervariable portion thereof (FAB, FAB″, etc.).

As used herein, “antigen binding fragment” refers to F(ab)2 or Fab fragments, which are composed of a light chain and the variable region of a heavy chain and are capable of binding an antigen with high affinity.

As used herein, “R. reniformis green fluorescent protein” or “R. reniformis GFP” refers to a polypeptide comprising the amino acid sequence of SEQ ID NO 2 or a fluorescent variant thereof. An R. reniformis GFP variant encompasses polypeptides of amino acid sequence SEQ ID NO: 2 that bear one or more mutations, including insertion or deletion of one or more amino acids, either at the N or C termini of the polypeptide or internal to the coding sequence. Variants of R. reniformis GFP retain the ability to emit light when excited by light within a given part of the spectrum, and may be excited by light of, or emit light in a portion of the spectrum that differs detectably from that which excites or which is emitted by wild-type R. reniformis GFP of amino acid sequence SEQ ID NO: 2. In addition to variants exhibiting different excitation or emission spectra, R. reniformis GFP variants include variants exhibiting increased fluorescence intensity relative to wild-type R. reniformis GFP. Preferably, a R. reniformis GFP, according to the invention, is encoded by a polynucleotide sequence comprising at least one humanized codon.

As used herein, “in frame” refers to the reading frame used for the translation of a fusion polypeptide nucleotide sequence. In a fusion polypeptide X-Y, coding sequences for polypeptide Y are said to be ‘in frame’ with upstream coding sequences for the polypeptide X if the translation of the coding sequences X-Y results in a fusion polypeptide wherein polypeptide X is fused to polypeptide Y.

As used herein, “wild-type R. reniformis GFP” refers to a polypeptide of SEQ ID NO: 2

As used herein, “identifying cells” refers to methods of identifying a GFP fluorescent cell within a population of cells. A “GFP-fluorescent cell”, as used herein, refers to a cell that expresses both a R. reniformis luciferase fusion polypeptide gene and a humanized R. reniformis GFP fusion polypeptide gene in a manner effective to result in the production of the R. reniformis GFP fusion protein in an amount sufficient to allow subsequent identification of the cell by detecting BRET-induced green fluorescence from GFP in the cell. GFP-fluorescent cells may be identified by a variety of methods, including microscopy and fluorescence activated cell sorting (FACS).

As used herein, “location” refers to the subcellular location of BRET-induced GFP fluorescence which, according to the invention, also indicates the subcellular location wherein a R. reniformis luciferase fusion protein binds to a R. reniformis GFP fusion protein.

As used herein, “receptor domain” refers to functional and structural entities within a receptor molecule. For example, cell surface receptors are comprised of an extracellular domain, a transmembrane domain and a cytoplasmic domain.

As used herein, “expression cassette”, in accordance with the present invention, refers to a recombinant vector wherein a gene of interest is operatively positioned downstream from a promoter wherein the promoter is capable of driving the expression of the gene in a living cell. An expression vector contains an expression cassette.

As used herein, “consisting essentially of” refers to the presence of R. reniformis luciferase and R. reniformis GFP in a composition, wherein they are the only proteins in the composition.

### BRIEF DESCRIPTION OF FIGURES

FIG. 1 is a graph demonstrating BRET ratios for 293 cells transfected with different plasmids.

FIG. 2 shows a the nucleotide sequence alignment between non-humanized (SEQ ID NO: 1) and humanized R. reniformis GFP (SEQ ID NO: 3). with the corresponding amino acid sequence depicted below the nucleotide sequence alignment (SEQ ID NO:2).

FIG. 3A shows a map of the R. reniformis phrGFP-N1 vector (Multiple cloning site (MCS) sequence:SEQ ID NO::27)

FIG. 3B shows a map of the R. reniformis phrGFP-C vector (Multiple cloning site (MCS) sequence:SEQ ID NO::28)

FIG. 4 shows the DNA sequence of the pRL-CMV (Promega) comprising R. reniformis luciferase (SEQ ID NO:4).

FIG. 5: Schematic description of a BRET assay

### DETAILED DESCRIPTION OF THE INVENTION

The invention describes the preparation and use of an “Renilla reniformis BRET system.” BRET or bioluminescence resonance energy transfer permits the analysis of protein-protein interactions both in vivo and in vitro by monitoring the non radiative transfer of energy from a bioluminescent donor (Luciferase) to a fluorescent acceptor (GFP or green fluorescent protein) in the presence of a substrate for luciferase.

The invention provides for methods of detecting protein:protein interactions, methods of determining the location of a protein:protein interaction, and a method of screening for a candidate modulator that increases or decreases the amount of a protein:protein interaction, wherein these methods utilize a first fusion protein comprising an R. reniformis GFP protein fused to a first polypeptide encoded by a humanized nucleotide sequence and a second fusion protein comprising a R. reniformis luciferase protein fused to a second polypeptide.

I. The Bioluminescence Resonance Energy Transfer (BRET) System

The invention provides for a BRET assay system wherein the energy transfer components of the system are derived from Renilla reniformis. According to the invention, independent protein domains that potentially complex with one another are respectively fused to R. reniformis luciferase and R. reniformis GFP.

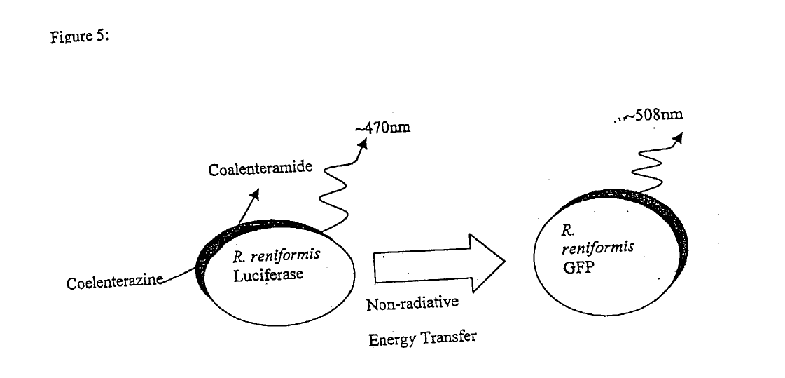

FIG. 5 illustrates the underlying principle of Bioluminescent Resonance Energy Transfer (BRET) and its use as a sensor: A) in isolation, a R. reniformis luciferase, emits blue light from the coelenterazine-derived chromophore; B) in isolation, a R. reniformis GFP that is excited with blue-green light emits green light from its integral peptide based fluorophore; C) when the luciferase and GFP associate as a complex in vivo or in vitro, the luciferase non-radiatively transfers its reaction energy to the GFP fluorophore, which then emits the green light; D) any molecular interaction that disrupts the luciferase-GFP complex can be quantitatively monitored by observing the spectral shift from green to blue light.

The nucleic acids, constructs and plasmids herein, permit preparation of a variety of configurations of fusion proteins that include an R. reniformis GFP, such as Renilla, with its cognate R. reniformis luciferase. Preferably, the GFP is fused to a first polypeptide domain and the luciferase is fused to a second polypeptide domain. Upon binding of the first and second polypeptide domains, the interaction of the luciferase with GFP will be altered thereby changing the emission signal of the complex.

A BRET assay is performed by any one of the following methods.

Cells are transfected as described herein below. Forty-eight hours posttransfection, adherent cells are detached with PBS/EDTA, or non-adherent cells are isolated by centrifugation, and cells are washed twice in PBS. Approximately 50,000 cells per well are distributed in a 96-well microplate (white Optiplate from Packard) in the presence or absence of isoproterenol (Sigma), propranolol (Sigma), or Mip-1α (Preprotech, Rocky Hill, N.J.). Coelenterazine is added at a final concentration of 5 μM, and readings are collected by using a modified topcount apparatus (BRETCount) that allows the sequential integration of the signals detected in the 440- to 500-nm and 510- to 590-nm windows (Angers et al., 2000, Proc. Natl. Acad Sci. USA, 97:3684-3689).

Alternatively, approximately 4×10⁶ cells in 1.5 ml of TEM buffer are added to a glass cuvette; an equal volume of TEM containing 10 μM coelenterazine is then added and the contents of the cuvette mixed. The emission spectrum (400-600 nM) is immediately acquired using a Spex fluorolog spectrofluorimeter with the excitation lamp turned off. For comparisons between experiments, emission spectra are normalized with the peak emission from Renilla luciferase in the region of 480 nm being defined as an intensity of 1.00. In some cases a BRET signal is calculated by measuring the area under the curve between 500 and 550 nm. Background is taken as the area of this region of the spectrum when examining emission from the isolated Renilla luciferase (McVey et al., 2001, J. Biol. Chem., 276:14092-14099).

In another embodiment, approximately 50,000 cells/well are distributed in a 96-well plate Coelenterazine (h form) (Molecular Probes, Inc., Eugene, Oreg.) is added to a final concentration of 5 μM, and readings are collected immediately following this addition. Repeated readings are taken for at least 5-10 min using a custom designed BRET instrument (Berthold, Australia) which allows sequential integration of the signals detected in the 440-500 and 510-590 nm windows. Data are represented as a normalized BRET ratio, which is defined as the BRET ratio for the co-expression of the Rluc and hrGFP constructs normalized against the BRET ratio for the Rluc expression construct alone. The BRET ratio is defined as ((emission at 510-590 nm)−(emission at 440-500 nm)×cf)/(emission at 440-500 nm), where cf corresponds to (emission at 510-590 nm/emission at 440-500 nm) for the Rluc construct expressed alone in the same experiment (Kroeger et al., 2001, J. Biol. Chem., 276:12736-12743).

The BRET assay described herein can be for used for screening protein:protein interactions in vitro or in vivo or in situ, including in cultured cells, tissues and animals.

II. How to Make R. reniformis Luciferase and GFP Fusion Polynucleotides According to the Invention.

A. Isolation of R. reniformis GFP cDNA Sequences

Methods for generating chimeric GFP and luciferase fusion proteins are described. The methods include linking a nucleic acid encoding a gene of interest, or portion thereof, to a nucleic acid encoding a GFP or luciferase provided herein in the same translational reading frame. The encoded-protein of interest may be linked in-frame to the amino- or carboxyl-terminus of the GFP or luciferase. The nucleic acid encoding the chimeric protein is then linked in operable association to a promoter element of a suitable expression vector.

1. R. reniformis cDNA Library Preparation.

Construction methods for libraries in a variety of different vectors, including, for example, bacteriophage, plasmids, and viruses capable of infecting eukaryotic cells are well known in the art. Any known library production method resulting in largely full-length clones of expressed genes may be used to provide a template for the isolation of GFP-encoding polynucleotides from R. Reniformis.

For the library used to isolate the luciferase and GFP-encoding polynucleotides disclosed herein, the following method was used. Poly(A) RNA was prepared from R. reniformis organisms as described by Chomczynski, P. and Sacchi, N. (1987, Anal. Biochem. 162: 156-159). cDNA was prepared using the ZAP-cDNA Synthesis Kit (Stratagene cat.#200400) according to the manufacturer's recommended protocols and inserted between the EcoR I and Xho I sites in the vector Lambda ZAP II. The resulting library contained 5×10⁶ individual primary clones, with an insert size range of 0.5-3.0 kb and an average insert size of 1.2 kb. The library was amplified once prior to use as template for PCR reactions.

2. Isolation of R. reniformis Coding Sequences by PCR.

cDNA sequences encoding R. reniformis GFP coding sequence are isolated by polymerase chain reaction (PCR) amplification of the sequence from within the cDNA library described herein. A large number of PCR methods are known to those skilled in the art. Thermal-cycled PCR (Mullis and Faloona, 1987, Methods Enzymol., 155: 335-350; see also, PCR Protocols, 1990, Academic Press, San Diego, Calif., USA for a review of PCR methods) uses multiple cycles of DNA replication catalyzed by a thermostable, DNA-dependent DNA polymerase to amplify the target sequence of interest. Briefly, oligonucleotide primers are selected such that they anneal on either side and on opposite strands of a sequence to be amplified. The primers are annealed and extended using a template-dependent thermostable DNA polymerase, followed by thermal denaturation and annealing of primers to both the original template sequence and the newly-extended template sequences, after which primer extension is performed. Repeating such cycles results in exponential amplification of the sequences between the two primers.

In addition to thermal cycled PCR, there are a number of other nucleic acid sequence amplification methods that may be used to amplify and isolate a GFP or Luciferase cDNA sequence. These include, for example, isothermal 3SR (Gingeras et al., 1990, Annales de Biologie Clinique, 48(7): 498-501; Guatelli et al., 1990, Proc. Natl. Acad. Sci. U.S.A., 87: 1874), and the DNA ligase amplification reaction (LAR), which permits the exponential increase of specific short sequences through the activities of any one of several bacterial; DNA ligases (Wu and Wallace, 1989, Genomics, 4: 560). The contents of both of these references are incorporated herein in their entirety by reference.

a. R. reniformis Luciferase cDNA Sequences

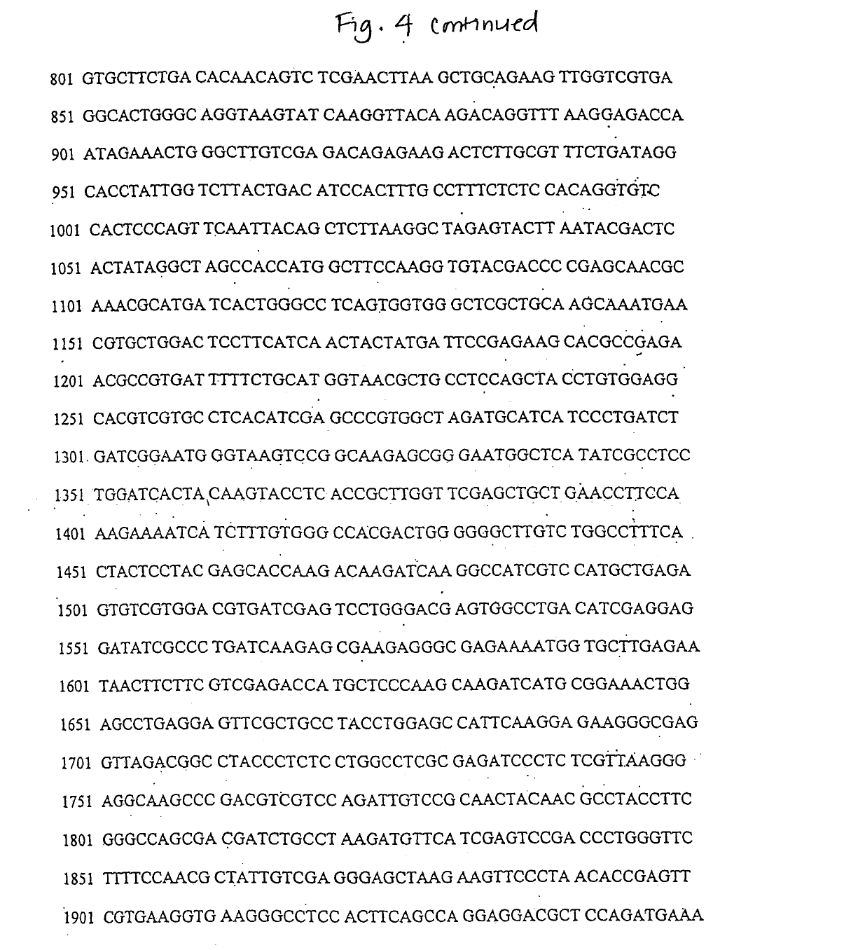

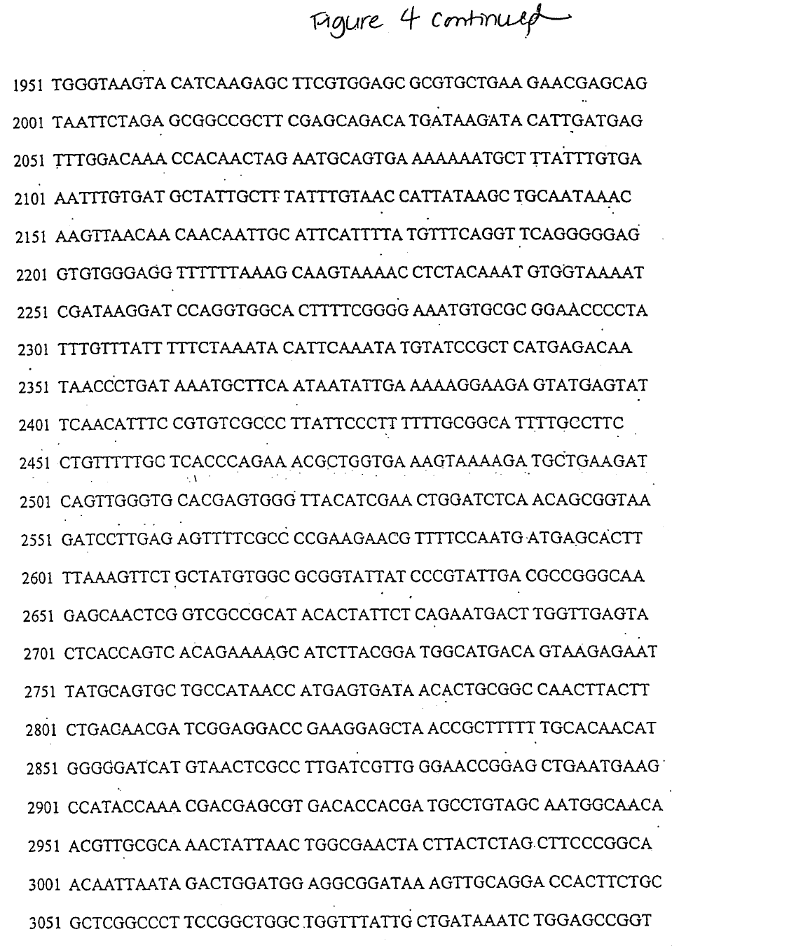

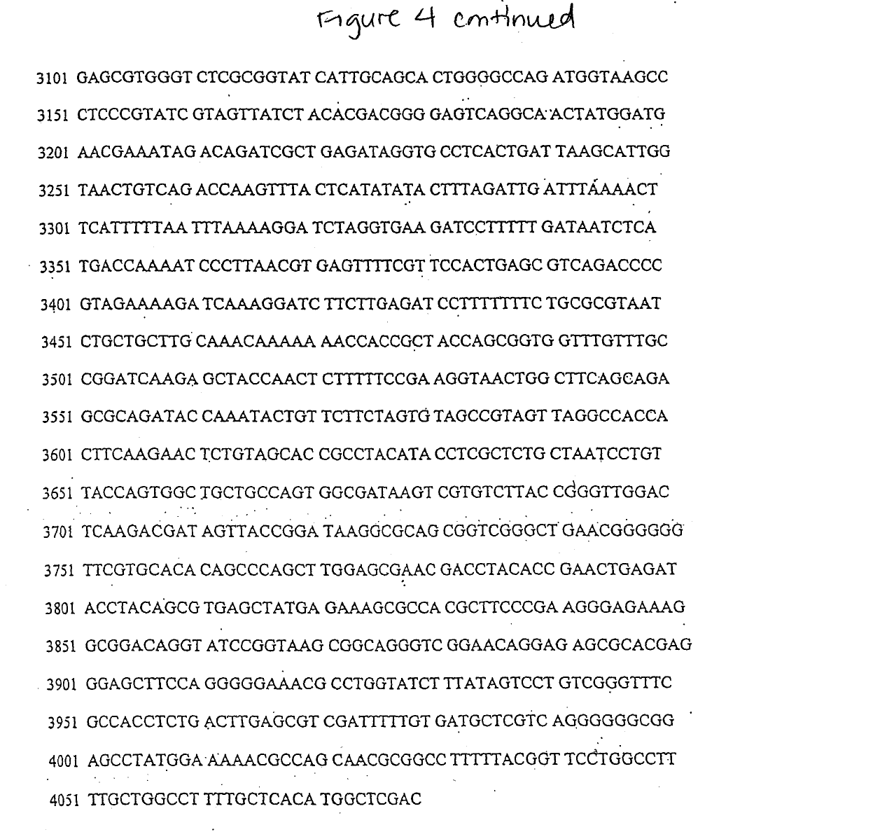

The cDNA sequence encoding Renilla reniformis luciferase (pRL-CMV, GenBank accession number: AF025843, see FIG. 4) can be purchased from Promega and was used as a template for all subsequent Renilla reniformis luciferase PCR reactions.

b. Isolation of R. reniformis GFP cDNA Sequences by PCR

To amplify a cDNA sequence encoding R. reniformis GFP from a R. reniformis cDNA plasmid or R. reniformis cDNA library, the following primers are used. The R. reniformis GFP coding sequence is amplified using the

| | |
|---|---|
| 5′ primer: | |
| 5′ AATTATTAGAATTCCGGGCCCGAGTGAGTAAACAAATATTGAAGAAC-3′ | (SEQ ID NO:5) |
| and the 3′ primer: | |
| 5′-ATAATATTCTCGAGTTAAACCCATTCGTGTAAGGATCC-3. | (SEQ ID NO:6) |

The 5′ primer contains EcoR I and NotI recognition sites to facilitate cloning of the amplified fragment. The 3′ primer contains a Xho I recognition site to facilitate subsequent cloning of the amplified fragment.

Oligonucleotides may be purchased from any of a number of commercial suppliers (for example, Life Technologies, Inc., Operon Technologies, etc.). Alternatively, oligonucleotide primers may be synthesized using methods well known in the art, including, for example, the phosphotriester (see Narang, S. A., et al., 1979, Meth. Enzymol., 68:90; and U.S. Pat. No. 4,356,270), phosphodiester (Brown, et al., 1979, Meth. Enzymol., 68:109), and phosphoramidite (Beaucage, 1993, Meth. Mol. Biol., 20:33) approaches. Each of these references is incorporated herein in its entirety by reference.

The typical PCR reaction is carried out in a 50 μl reaction volume containing 1×TaqPlus Precision buffer (Stratagene), 250 μM of each dNTP, 200 nM of each PCR primer, 2.5 U TaqPlus Precision enzyme (Stratagene) and 1-10 ng of cDNA template. Reactions are carried out in a Robocycler Gradient 40 (Stratagene) as follows: 1 min at 95° C. (1 cycle), 1 min at 95° C., 1 min at 50-55° C., 1 min at 72° C. (40 cycles), and 1 min at 72 C. (1 cycle). Reaction products are then resolved on a 1% agarose gel, and a PCR product of the predicted size is excised and purified using the StrataPrep DNA Gel Extraction Kit (Stratagene). Other methods of isolating and purifying amplified nucleic acid fragments are well known to those skilled in the art. The PCR fragment is then subcloned into an appropriate recombinant vector (see below). Both strands of the cloned GFP or Luciferase fragment are then completely sequenced.

c. Generation of Humanized GFP cDNA Sequences

i. Humanized Codon Usage

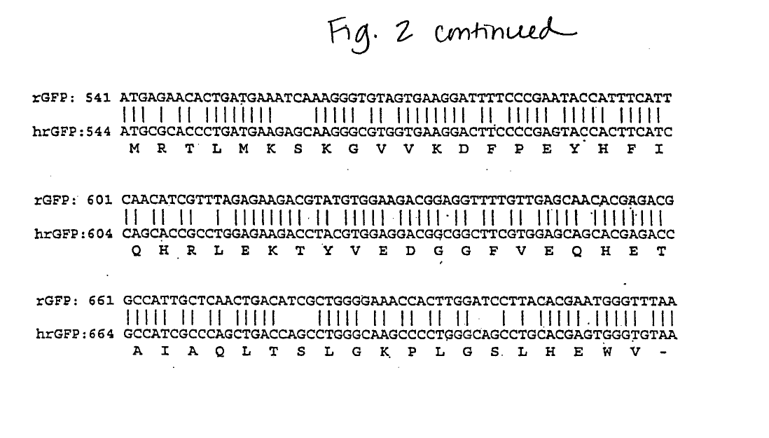

The DNA sequence encoding wild-type R. reniformis GFP is modified to enhance its expression in mammalian or human cells (see FIG. 2). The codon usage of R. reniformis is optimal for expression in R. Reniformis, but not for expression in mammalian or human systems. Therefore, the adaptation of the sequence isolated from the sea pansy for expression in higher eukaryotes involves the modification of specific codons to change those less favored in mammalian or human systems to those more commonly used in these systems. This so-called “humanization” is accomplished by site-directed mutagenesis of the less favored codons as described herein or as known in the art. Similar modifications of the A. victoria GFP coding sequences are described in U.S. Pat. No. 5,874,304. The preferred codons for human gene expression are listed in Table 1. The codons in the table are arranged from left to right in descending order of relative use in human genes. Consideration of the codons in R. reniformis GFP (SEQ ID NO: 1) relative to those favored in human genes allows one of skill in the art to identify which codons to modify in the R. reniformis GFP gene to achieve more efficient expression in human or mammalian cells. In particular, those codons underlined in the table are almost never used in known human genes and, if found in the R. reniformis sequence, would therefore represent the most important codons to modify for enhanced expression efficiency in mammalian or human cells.

TABLE 1
PREFERRED DNA CODONS FOR HUMAN USE

| | | | | SEQ ID |
|---|---|---|---|---|
| Amino Acids | | | Codons Preferred in Human Genes | NO |
| Alanine | Ala | A | GCC GCT GCA GCG | 7 |
| Cysteine | Cys | C | TGT TGT | 8 |
| Aspartic acid | Asp | D | GAC GAT | 9 |
| Glutamic acid | Glu | E | GAG GAA | 10 |
| Phenylalanine | Phe | F | TTC TTT | 11 |
| Glycine | Gly | G | GGC GGG GGA GGT | 12 |
| Histidine | His | H | CAC CAT | 13 |
| Isoleucine | Ile | I | ATC ATT ATA | 14 |
| Lysine | Lys | K | AAG AAA | 15 |
| Leucine | Leu | L | CTG TTG CTT CTA TTA | 16 |
| Methionine | Met | M | ATG | 17 |
| Asparagine | Asn | N | AAC AAT | 18 |
| Proline | Pro | P | CCC CCT CCA CCG | 19 |
| Glutamine | Gln | Q | CAG CAA | 20 |
| Arginine | Arg | R | CGC AGG CGG AGA CGA CGT | 21 |
| Serine | Ser | S | AGC TCC TCT AGT TCA TCG | 22 |
| Threonine | Thr | T | ACC ACA ACT ACG | 23 |
| Valine | Val | V | GTG GTC GTT GTA | 24 |
| Tryprophan | Trp | W | TGG | 25 |
| Tyrosine | Tyr | Y | TAC TAT | 26 |

The codons at the left represent those most preferred for use in human genes, with human usage decreasing towards the right. Underlined codons are almost never used in human genes and are therefore not preferred.

ii. Site-Directed or Targeted Mutagenesis

There are a number of site-directed mutagenesis methods known in the art which allow one to mutate a particular site or region in a straightforward manner. These methods are embodied in a number of kits available commercially for the performance of site-directed mutagenesis, including both conventional and PCR-based methods. Examples include the EXSITE™ PCR-based site-directed mutagenesis kit available from Stratagene (Catalog No. 200502; PCR based) and the QUIKCHANGE™ site-directed mutagenesis kit from Stratagene (Catalog No. 200518; PCR based), and the CHAMELEON® double-stranded site-directed mutagenesis kit, also from Stratagene (Catalog No. 200509).

Older methods of site-directed mutagenesis known in the art relied upon sub-cloning of the sequence to be mutated into a vector, such as an M13 bacteriophage vector, that allows the isolation of single-stranded DNA template. In these methods one annealed a mutagenic primer (i.e., a primer capable of annealing to the site to be mutated but bearing one or more mismatched nucleotides at the site to be mutated) to the single-stranded template and then polymerized the complement of the template starting from the 3′ end of the mutagenic primer. The resulting duplexes were then transformed into host bacteria and plaques were screened for the desired mutation.

More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

The protocol described below accommodates these considerations through the following steps. First, the template concentration used is approximately 1000-fold higher than that used in conventional PCR reactions, allowing a reduction in the number of cycles from 25-30 down to 5-10 without dramatically reducing product yield. Second, the restriction endonuclease DpnI (recognition target sequence: 5-Gm6ATC-3 (SEQ ID NO: 29), where the A residue is methylated) is used to select against parental DNA, since most common strains of E. coli Dam methylate their DNA at the sequence 5′-GATC-3′ (SEQ ID NO: 30). Third, Taq Extender is used in the PCR mix in order to increase the proportion of long (i.e., full plasmid length) PCR products. Finally, Pfu DNA polymerase is used to polish the ends of the PCR product prior to intramolecular ligation using T4 DNA ligase.

The method is described in detail as follows:

Plasmid template DNA (approximately 0.5 pmole) is added to a PCR cocktail containing: 1×mutagenesis buffer (20 mM Tris HCl, pH 7.5; 8 mM MgCl₂; 40 ug/ml BSA); 12-20 pmole of each primer (one of skill in the art may design a mutagenic primer as necessary, giving consideration to those factors such as base composition, primer length and intended buffer salt concentrations that affect the annealing characteristics of oligonucleotide primers; one primer must contain the desired mutation, and one (the same or the other) must contain a 5′ phosphate to facilitate later ligation), 250 uM each dNTP, 2.5 U Taq DNA polymerase, and 2.5 U of Taq Extender (Available from Stratagene; See Nielson et al. (1994) Strategies 7: 27, and U.S. Pat. No. 5,556,772). The PCR cycling is performed as follows: 1 cycle of 4 min at 94° C., 2 min at 50° C. and 2 min at 72° C.; followed by 5-10 cycles of 1 min at 94° C., 2 min at 54° C. and 1 min at 72° C. The parental template DNA and the linear, PCR-generated DNA incorporating the mutagenic primer are treated with DpnI (10 U) and Pfu DNA polymerase (2.5 U). This results in the DpnI digestion of the in vivo methylated parental template and hybrid DNA and the removal, by Pfu DNA polymerase, of the non-template-directed Taq DNA polymerase-extended base(s) on the linear PCR product. The reaction is incubated at 37° C. for 30 min and then transferred to 72° C. for an additional 30 min. Mutagenesis buffer (115 ul of 1×) containing 0.5 mM ATP is added to the DpnI-digested, Pfu DNA polymerase-polished PCR products. The solution is mixed and 10 ul are removed to a new microfuge tube and T4 DNA ligase (2-4 U) is added. The ligation is incubated for greater than 60 min at 37° C. Finally, the treated solution is transformed into competent E. coli according to standard methods.

3. Generation of R. reniformis luciferase and R. reniformis GFP Fusion Polynucleotides

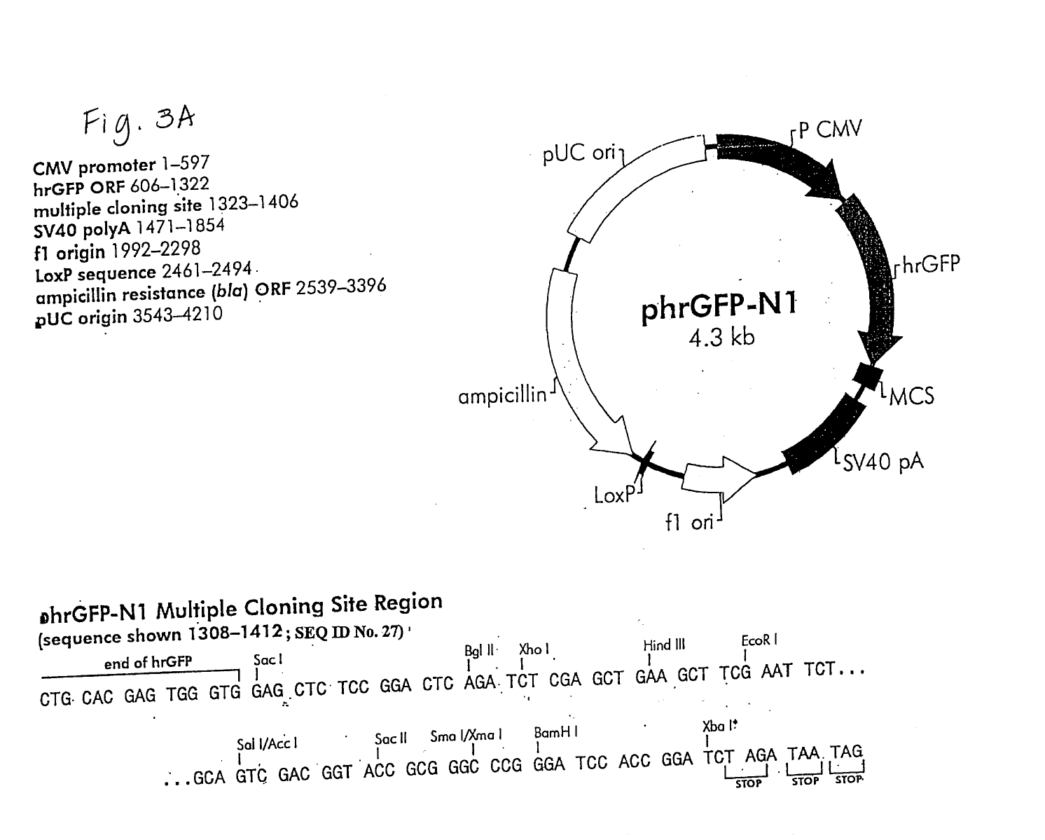

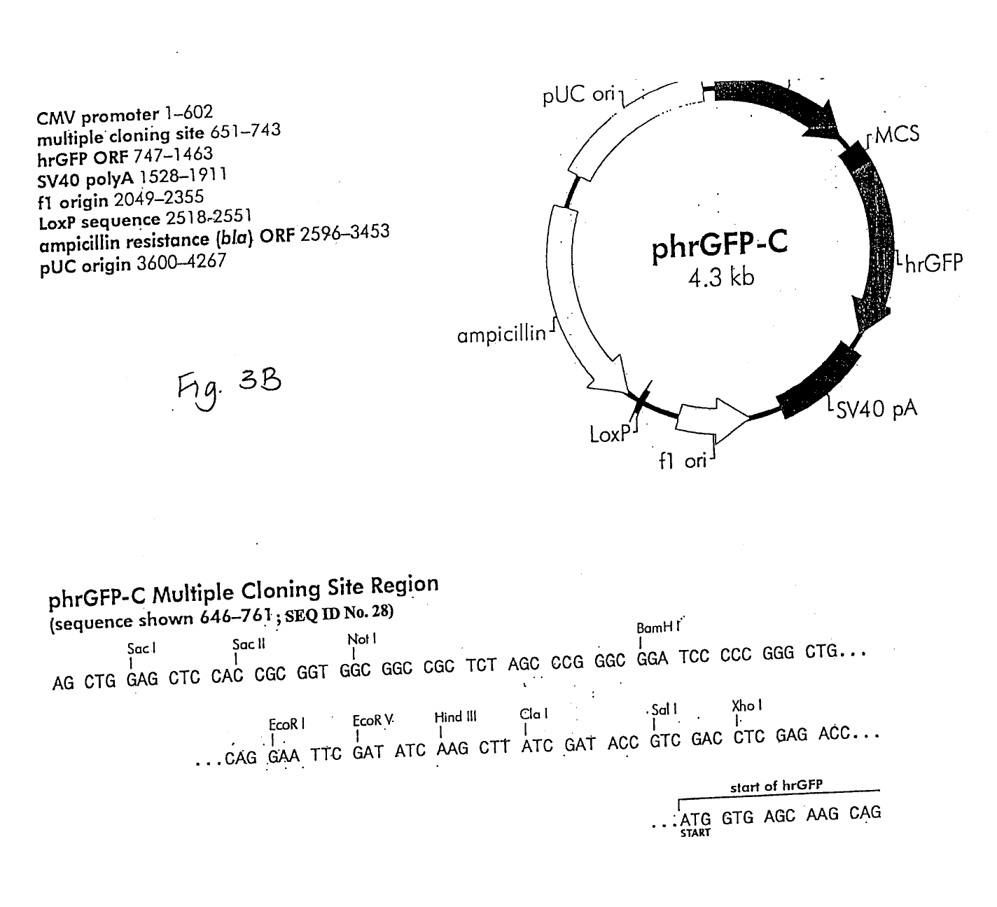

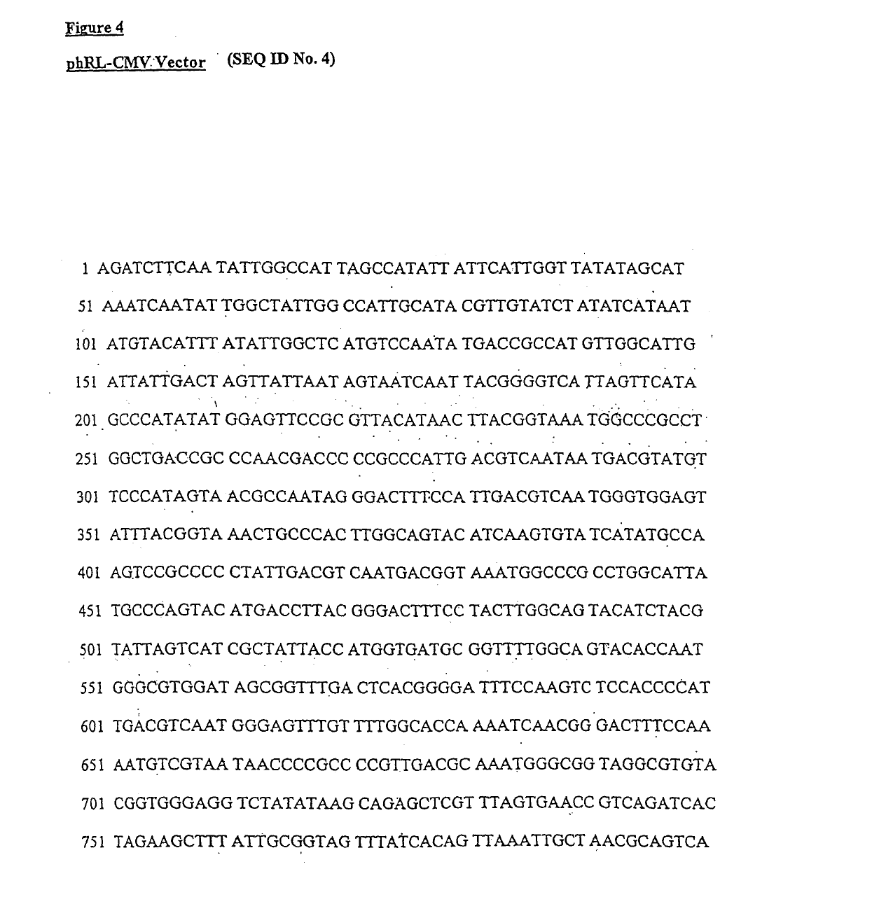

cDNA sequences encoding R. reniformis luciferase (see FIG. 4) and cDNA sequences encoding R. reniformis GFP (see FIG. 2), according to the invention, are fused in frame to polynucleotide sequences encoding polypeptide domains of interest, via cloning methods well-known in the art (Sambrook et al., 1989, Molecular Cloning, A Laboratory Manual, Cold Spring Harbor Laboratory Press, Cold Spring Harbor, N.Y., and Current Protocols in Molecular Biology, Ausubel, F. M. et al. (eds.) Greene Publishing Associates, (1989)). It is assumed that one of skill in the art can, given the polynucleotide sequences disclosed herein or those accessible on Genbank, readily construct genes comprising i) a polynucleotide sequence encoding R. reniformis Luciferase fused in frame with a sequence comprising one or more polypeptides or polypeptide domains of interest and ii) a humanized polynucleotide sequence encoding R. reniformis GFP fused in frame with a sequence comprising one or more polypeptides or polypeptide domains of interest. As used herein, the term “polypeptide of interest” or “domain of interest” refers to any polypeptide or polypeptide domain one wishes to fuse to either a R. reniformis luciferase or R. reniformis GFP molecule of the invention. Again, the polynucleotide sequences encoding the polypeptide domains of interest are isolated by PCR or by isolation of a restriction enzyme digested DNA fragment according to recombinant DNA techniques well known to a person of the art. The fusion of R. reniformis luciferase or R. reniformis GFP polypeptide of the invention with a polypeptide of interest is made through linkage of the R. reniformis luciferase or humanized R. reniformis GFP coding sequence to either the N or C terminus of the fusion partner, according to methods well-known in the art. Mammalian expression vectors comprising humanized R. reniformis GFP (phrGFP-N1 and phrGFP-C, see FIG. 3) are commercially available and are described in Stratagene's online newsletter (B. Rogers et al., Strategies 13, 141-144 (2000)). The use of R. reniformis phrGFP-N1 and R. reniformis phrGFP-C vectors including detailed cloning procedures are available from Stratagene, La Jolla, Calif. 92037.

4. Generation of R. reniformis Luciferase and GFP Fusion Polypeptides.

The production of R. reniformis luciferase and R. reniformis GFP fusion proteins from recombinant vectors comprising luciferase-encoding and GFP-encoding polynucleotides of the invention may be effected in a number of ways known to those skilled in the art. For example, plasmids, bacteriophage or viruses may be introduced to prokaryotic or eukaryotic cells by any of a number of ways known to those skilled in the art. Useful vectors, cells, methods of introducing vectors to cells and methods of detecting and isolating R. reniformis luciferase and R. reniformis GFP fusion proteins are also described herein below.

A. Vectors Useful According to the Invention.

There is a wide array of vectors known and available in the art that are useful for the expression of R. reniformis luciferase and R. reniformis GFP fusion proteins according to the invention. The selection of a particular vector clearly depends upon the intended use of the R. reniformis luciferase and GFP fusion proteins. For example, the selected vector must be capable of driving expression of the fusion protein in the desired cell type, whether that cell type be prokaryotic or eukaryotic. Many vectors comprise sequences allowing both prokaryotic vector replication and eukaryotic expression of operably linked gene sequences.

Vectors useful according to the invention may be autonomously replicating, that is, the vector, for example, a plasmid, exists extrachromosomally and its replication is not necessarily directly linked to the replication of the host cell's genome. Alternatively, the replication of the vector may be linked to the replication of the host's chromosomal DNA, for example, the vector may be integrated into the chromosome of the host cell as achieved by retroviral vectors and in stably transfected cell lines.

Vectors useful according to the invention preferably comprise sequences operably linked to R. reniformis fusion protein coding sequences that permit the transcription and translation of fusion protein polynucleotide sequences. “R. reniformis fusion proteins” according to the invention means either an R. reniformis luciferase fusion protein or an R. reniformis GFP fusion protein as described herein. Sequences that permit the transcription of the linked R. reniformis fusion protein sequences include a promoter and optionally also include an enhancer element or elements permitting the strong expression of the linked sequences. The term “transcriptional regulatory sequences” refers to the combination of a promoter and any additional sequences conferring desired expression characteristics (e.g., high level expression, inducible expression, tissue- or cell-type-specific expression) on an operably linked nucleic acid sequence.

An “expression vector”, according to the invention, comprises either a) a constitutive promoter, such as viral promoters or promoters from mammalian genes that are generally active in promoting transcription. Examples of constitutive viral promoters include the HSV, TK, RSV, SV40 and CMV promoters, of which the CMV promoter is a currently preferred example. Examples of constitutive mammalian promoters include various housekeeping gene promoters, as exemplified by the β-actin promoter; or b) Inducible promoters and/or regulatory elements are also contemplated for use with the expression vectors of the invention. Examples of suitable inducible promoters include promoters from genes such as cytochrome P450 genes, heat shock protein genes, metallothionein genes, hormone-inducible genes, such as the estrogen gene promoter, and the like. Promoters that are activated in response to exposure to ionizing radiation, such as fos, jun and egr-1, are also contemplated. The tetVP16 promoter that is responsive to tetracycline is a currently preferred example; or c) Tissue-specific promoters are also contemplated for use with the expression vectors of the invention. Examples of such promoters that may be used with the expression vectors of the invention include promoters from the liver fatty acid binding (FAB) protein gene, specific for colon epithelial cells; the insulin gene, specific for pancreatic cells; the transphyretin, α1-antitrypsin, plasminogen activator inhibitor type 1 (PAI-1), apolipoprotein AI and LDL receptor genes, specific for liver cells; the myelin basic protein (MBP) gene, specific for oligodendrocytes; the glial fibrillary acidic protein (GFAP) gene, specific for glial cells; OPSIN, specific for targeting to the eye; and the neural-specific enolase (NSE) promoter that is specific for nerve cells.

The selected promoter may be any DNA sequence that exhibits transcriptional activity in the selected host cell, and may be derived from a gene normally expressed in the host cell or from a gene normally expressed in other cells or organisms. Examples of promoters include, but are not limited to the following: A) prokaryotic promoters—E. coli lac, tac, or trp promoters, lambda phage P_{R} or P_{L} promoters, bacteriophage T7, T3, Sp6 promoters, B. subtilis alkaline protease promoter, and the B. stearothermophilus maltogenic amylase promoter, etc.; B) eukaryotic promoters—yeast promoters, such as GAL1, GAL4 and other glycolytic gene promoters (see for example, Hitzeman et al., 1980, J. Biol. Chem. 255: 12073-12080; Alber & Kawasaki, 1982, J. Mol. Appl. Gen. 1: 419-434), LEU2 promoter (Martinez-Garcia et al., 1989, Mol Gen Genet. 217: 464-470), alcohol dehydrogenase gene promoters (Young et al., 1982, in Genetic Engineering of Microorganisms for Chemicals, Hollaender et al., eds., Plenum Press, NY), or the TPI1 promoter (U.S. Pat. No. 4,599,311); insect promoters, such as the polyhedrin promoter (U.S. Pat. No. 4,745,051; Vasuvedan et al., 1992, FEBS Lett. 311: 7-11), the P10 promoter (Vlak et al., 1988, J. Gen. Virol. 69: 765-776), the Autographa californica polyhedrosis virus basic protein promoter (EP 397485), the baculovirus immediate-early gene 1 promoter (U.S. Pat. Nos. 5,155,037 and 5,162,222), the baculovirus 39K delayed-early gene promoter (also U.S. Pat. Nos. 5,155,037 and 5,162,222) and the OpMNPV immediate early promoter 2; mammalian promoters—the SV40 promoter (Subramani et al., 1981, Mol. Cell. Biol. 1: 854-864), metallothionein promoter (MT-1; Palmiter et al., 1983, Science 222: 809-814), adenovirus 2 major late promoter (Yu et al.,1984, Nucl. Acids Res. 12: 9309-21), cytomegalovirus (CMV) or other viral promoter (Tong et al., 1998, Anticancer Res. 18: 719-725), or even the endogenous promoter of a gene of interest in a particular cell type.

A selected promoter may also be linked to sequences rendering it inducible or tissue-specific. For example, the addition of a tissue-specific enhancer element upstream of a selected promoter may render the promoter more active in a given tissue or cell type. Alternatively, or in addition, inducible expression may be achieved by linking the promoter to any of a number of sequence elements permitting induction by, for example, thermal changes (temperature sensitive), chemical treatment (for example, metal ion- or IPTG-inducible), or the addition of an antibiotic inducing agent (for example, tetracycline). Inducible expression of R. reniformis luciferase and hrGFP fusion polypeptides may be particularly desirable because of the known cytotoxicity of GFP proteins and its variants.

Regulatable expression is achieved using, for example, expression systems that are drug inducible (e.g., tetracycline, rapamycin or hormone-inducible). Drug-regulatable promoters that are particularly well suited for use in mammalian cells include the tetracycline regulatable promoters, and glucocorticoid steroid-, sex hormone steroid-, ecdysone-, lipopolysaccharide (LPS)- and isopropylthiogalactoside (IPTG)-regulatable promoters. A regulatable expression system for use in mammalian cells should ideally, but not necessarily, involve a transcriptional regulator that binds (or fails to bind) non mammalian DNA motifs in response to a regulatory agent, and a regulatory sequence that is responsive only to this transcriptional regulator.

One inducible expression system that is well suited for the regulated expression of a luciferase or hrGFP polypeptide of the invention or variant thereof, is the tetracycline-regulatable expression system, which is founded on the efficiency of the tetracycline resistance operon of E. coli. The binding constant between tetracycline and the tet repressor is high while the toxicity of tetracycline for mammalian cells is low, thereby allowing for regulation of the system by tetracycline concentrations in eukaryotic cell culture or within a mammal that do not affect cellular growth rates or morphology. Binding of the tet repressor to the operator occurs with high specificity.

Versions of the tet-regulatable system exist that allow either positive or negative regulation of gene expression by tetracycline. In the absence of tetracycline or a tetracycline analog, the wild-type bacterial tet repressor protein causes negative regulation of genes driven by promoters containing repressor binding elements from the tet operator sequences. Gossen & Bujard (1995, Science 268: 1766-1769; also International patent application No. WO 96/01313) describe a tet-regulatable expression system that exploits this positive regulation by tetracycline. In this system, tetracycline binds to a tet repressor fusion protein, rtTA, and prevents it from binding to the tet operator DNA sequence, thus allowing transcription and expression of the linked gene only in the presence of the drug.

This positive tetracycline-regulatable system provides one means of stringent temporal regulation of the R. reniformis fusion proteins of the invention (Gossen & Bujard, 1995, supra). The tet operator (tet O) sequence is now well known to those skilled in the art. For a review, the reader is referred to Hillen & Wissmann (1989) in Protein-Nucleic Acid Interaction, “Topics in Molecular and Structural Biology”, eds. Saenger & Heinemann, (Macmillan, London), Vol. 10, pp 143-162. Typically the nucleic acid sequence encoding the GFP polypeptide is placed downstream of a plurality of tet O sequences: generally 5 to 10 such tet O sequences are used, in direct repeats.

In addition to the tetracycline-regulatable systems, a number of other options exist for the regulated or inducible expression of R. reniformis fusion proteins according to the invention. For example, the E. coli lac promoter is responsive to lac repressor (lacI) DNA binding at the lac operator sequence. The elements of the operator system are functional in heterologous contexts, and the inhibition of lacI binding to the lac operator by IPTG is widely used to provide inducible expression in both prokaryotic, and more recently, eukaryotic cell systems. In addition, the rapamycin-controlled transcriptional activator system described by Rivera et al. (1996, Nature Med. 2: 1028-1032) provides transcriptional activation dependent on rapamycin. That system has low baseline expression and a high induction ratio.

Another option for regulated or inducible expression of R. reniformis fusion proteins involves the use of a heat-responsive promoter. Activation is induced by incubation of cells, transfected with a R. reniformis fusion protein construct regulated by a temperature-sensitive transactivator, at the permissive temperature prior to administration. For example, transcription regulated by a co-transfected, temperature sensitive transcription factor active only at 37° C. may be used if cells are first grown at, for example, 32° C., and then switched to 37° C. to induce expression.

Tissue-specific promoters may also be used in R. reniformis fusion protein constructs of the invention. A wide variety of tissue-specific promoters is known. As used herein, the term “tissue-specific” means that a given promoter is transcriptionally active (i.e., directs the expression of linked sequences sufficient to permit detection of the polypeptide product of the promoter) in less than all cells or tissues of an organism. A tissue specific promoter is preferably active in only one cell type, but may, for example, be active in a particular class or lineage of cell types (e.g., hematopoietic cells). A tissue specific promoter useful according to the invention comprises those sequences necessary and sufficient for the expression of an operably linked nucleic acid sequence in a manner or pattern that is essentially the same as the manner or pattern of expression of the gene linked to that promoter in nature. The following is a non-exclusive list of tissue specific promoters and literature references containing the necessary sequences to achieve expression characteristic of those promoters in their respective tissues; the entire content of each of these literature references is incorporated herein by reference. Examples of promoters useful for the tissue specific expression of R. reniformis fusion proteins of the invention are as follows:

Bowman et al., 1995 Proc. Natl. Acad. Sci. USA 92,12115-12119 describe a brain-specific transferrin promoter; the synapsin I promoter is neuron specific (Schoch et al., 1996 J. Biol. Chem. 271, 3317-3323); the necdin promoter is post-mitotic neuron specific (Uetsuki et al., 1996 J. Biol. Chem. 271, 918-924); the neurofilament light promoter is neuron specific (Charron et al., 1995 J. Biol. Chem. 270, 30604-30610); the acetylcholine receptor promoter is neuron specific (Wood et al., 1995 J. Biol. Chem. 270, 30933-30940); the potassium channel promoter is high-frequency firing neuron specific (Gan et al., 1996 J. Biol. Chem 271, 5859-5865); the chromogranin A promoter is neuroendocrine cell specific (Wu et al., 1995 A.J. Clin. Invest. 96, 568-578); the Von Willebrand factor promoter is brain endothelium specific (Aird et al., 1995 Proc. Natl. Acad. Sci. USA 92, 4567-4571); the flt-1 promoter is endothelium specific (Morishita et al., 1995 J. Biol. Chem. 270, 27948-27953); the preproendothelin-1 promoter is endothelium, epithelium and muscle specific (Harats et al., 1995 J. Clin. Invest. 95, 1335-1344); the GLUT4 promoter is skeletal muscle specific (Olson and Pessin, 1995 J. Biol. Chem. 270, 23491-23495); the Slow/fast troponins promoter is slow/fast twitch myofibre specific (Corin et al., 1995 Proc. Natl. Acad. Sci. USA 92, 6185-6189); the α-Actin promoter is smooth muscle specific (Shimizu et al., 1995 J. Biol. Chem. 270, 7631-7643); the Myosin heavy chain promoter is smooth muscle specific (Kallmeier et al., 1995 J. Biol. Chem. 270, 30949-30957); the E-cadherin promoter is epithelium specific (Hennig et al., 1996 J. Biol. Chem. 271, 595-602); the cytokeratins promoter is keratinocyte specific (Alexander et al., 1995 B. Hum. Mol. Genet. 4, 993-999); the transglutaminase 3 promoter is keratinocyte specific (J. Lee et al., 1996 J. Biol. Chem. 271, 4561-4568); the bullous pemphigoid antigen promoter is basal keratinocyte specific (Tamai et al., 1995 J. Biol. Chem. 270, 7609-7614); the keratin 6 promoter is proliferating epidermis specific (Ramirez et al., 1995 Proc. Natl. Acad. Sci. USA 92, 4783-4787); the collagen α1 promoter is hepatic stellate cell and skin/tendon fibroblast specific (Houglum et al., 1995 J. Clin. Invest. 96, 2269-2276); the type X collagen promoter is hypertrophic chondrocyte specific (Long & Linsenmayer, 1995 Hum. Gene Ther. 6, 419-428); the Factor VII promoter is liver specific (Greenberg et al., 1995 Proc. Natl. Acad. Sci. USA 92, 12347-1235); the fatty acid synthase promoter is liver and adipose tissue specific (Soncini et al., 1995 J. Biol. Chem. 270, 30339-3034); the carbamoyl phosphate synthetase I promoter is portal vein hepatocyte and small intestine specific (Christoffels et al., 1995 J. Biol. Chem. 270, 24932-24940); the Na—K—Cl transporter promoter is kidney (loop of Henle) specific (Igarashi et al., 1996 J. Biol. Chem. 271, 9666-9674); the scavenger receptor A promoter is macrophages and foam cell specific (Horvai et al., 1995 Proc. Natl. Acad. Sci. USA 92, 5391-5395); the glycoprotein IIb promoter is megakaryocyte and platelet specific (Block & Poncz, 1995 Stem Cells 13, 135-145); the yc chain promoter is hematopoietic cell specific (Markiewicz et al., 1996 J. Biol. Chem. 271, 14849-14855); and the CD11b promoter is mature myeloid cell specific (Dziennis et al., 1995 Blood 85, 319-329).

Any tissue specific transcriptional regulatory sequence known in the art may be used with a vector encoding R. reniformis fusion proteins according to the invention.

In addition to promoter/enhancer elements, vectors useful according to the invention may further comprise a suitable terminator. Such terminators include, for example, the human growth hormone terminator (Palmiter et al., 1983, supra), or, for yeast or fungal hosts, the TPI1 (Alber & Kawasaki, 1982, supra) or ADH3 terminator (McKnight et al., 1985, EMBO J. 4: 2093-2099).

Vectors useful according to the invention may also comprise polyadenylation sequences (e.g., the SV40 or Ad5E1b poly(A) sequence), and translational enhancer sequences (e.g., those from Adenovirus VA RNAs). Further, a vector useful according to the invention may encode a signal sequence directing the recombinant polypeptide to a particular cellular compartment or, alternatively, may encode a signal directing secretion of the recombinant polypeptide.

Coordinate expression of R. reniformis luciferase and R. reniformis GFP from the same promoter in a recombinant vector may be achieved by using an IRES element, such as the internal ribosomal entry site of Poliovirus type 1 from pSBC-1 (Dirks et al., 1993, Gene 128:247-9). Internal ribosome binding site (IRES) elements are used to create multigenic or polycistronic messages. IRES elements are able to bypass the ribosome scanning mechanism of 5′ methylated Cap-dependent translation and begin translation at internal sites (Pelletier and Sonenberg, 1988, Nature 334: 320-325). IRES elements from two members of the picanovirus family (polio and encephalomyocarditis) have been described (Pelletier and Sonenberg, 1988, supra), as well an IRES from a mammalian message (Macejak and Sarnow, 1991 Nature 353: 90-94). Any of the foregoing may be used in a R. reniformis luciferase and R. reniformis GFP vector in accordance with the present invention.

IRES elements can also be linked to heterologous open reading frames. Multiple open reading frames can be transcribed together, each separated by an IRES, creating polycistronic messages. By virtue of the IRES element, each open reading frame is accessible to ribosomes for efficient translation. In this manner, R. reniformis luciferase and R. reniformis GFP fusion protein genes, can be efficiently expressed using a single promoter/enhancer to transcribe a single message. Alternatively any heterologous open reading frame can be linked to IRES elements. In the present context, this means any selected protein that one desires to express and any selectable marker gene. For instance, the R. reniformis luciferase fusion protein gene can be co-expressed with a gene conferring resistance to hygromycin and the R. reniformis GFP fusion protein gene could be co-expressed with a gene conferring resistance to neomycin. In this way, the expression of multiple proteins can be achieved in the same cell.

A vector useful according to the invention may also comprise a selectable marker allowing identification of a cell that has received functional copies of both R. reniformis luciferase and R. reniformis GFP-derived fusion protein gene constructs. In its simplest form, the R. reniformis GFP sequence itself, linked to a chosen promoter may be considered a selectable marker, in that illumination of cells or cell lysates with the proper wavelength of light and measurement of emitted fluorescence at the expected wavelength allows detection of cells that express the R. reniformis GFP construct. Likewise, the transfected cells can be incubated with a substrate for luciferase, for example coelentrazine, and luciferase mediated bioluminescence can be detected. These control assays can be used to ensure that 1) a cell harbors both an R. reniformis luciferase fusion protein gene expression vector and an R. reniformis GFP fusion protein gene expression vector and 2) R. reniformis luciferase and R. reniformis GFP moieties of each R. reniformis fusion protein are biologically active. In other forms, the selectable marker may comprise an antibiotic resistance gene, such as the neomycin, bleomycin, zeocin or phleomycin resistance genes, or it may comprise a gene whose product complements a defect in a host cell, such as the gene encoding dihydrofolate reductase (DHFR), or, for example, in yeast, the Leu2 gene.

a. Plasmid Vectors.

Any plasmid vector that allows expression of a R. reniformis luciferase or R. reniformis GFP coding sequence of the invention in a selected host cell type is acceptable for use according to the invention. A plasmid vector useful in the invention may have any or all of the above-noted characteristics of vectors useful according to the invention. Plasmid vectors useful according to the invention include, but are not limited to the following examples: Bacterial—pQE70, pQE60, pQE-9 (Qiagen) pBs, phagescript, psiX174, pBluescript SK, pBsKS, pNH8a, pNH16a, pNH18a, pNH46a (Stratagene); pTrc99A, pKK223-3, pKK233-3, pDR540, and pRIT5 (Pharmacia); Eukaryotic—pWLneo, pSV2cat, pOG44, pXT1, pSG (Stratagene) pSVK3, pBPV, pMSG, and pSVL (Pharmacia). However, any other plasmid or vector may be used as long as it is replicable and viable in the host.

b. Bacteriophage Vectors.

There are a number of well known bacteriophage-derived vectors useful according to the invention. Foremost among these are the lambda-based vectors, such as Lambda Zap II or Lambda-Zap Express vectors (Stratagene) that allow inducible expression of the polypeptide encoded by the insert. Others include filamentous bacteriophage such as the M13-based family of vectors.

c. Viral Vectors.

A number of different viral vectors are useful according to the invention, and any viral vector that permits the introduction and expression of sequences encoding R. reniformis luciferase and hrGFP fusion polypeptides or variants thereof in cells is acceptable for use in the methods of the invention. Viral vectors that can be used to deliver foreign nucleic acid into cells include but are not limited to retroviral vectors, adenoviral vectors, adeno-associated viral vectors, herpesviral vectors, and Semiliki forest viral (alphaviral) vectors. Defective retroviruses are well characterized for use in gene transfer (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14, and other standard laboratory manuals.

In addition to retroviral vectors, Adenovirus can be manipulated such that it encodes and expresses a gene product of interest but is inactivated in terms of its ability to replicate in a normal lytic viral life cycle (see for example Berkner et al., 1988, BioTechniques 6:616; Rosenfeld et al., 1991, Science 252:431-434; and Rosenfeld et al., 1992, Cell 68:143-155). Suitable adenoviral vectors derived from the adenovirus strain Ad type 5 d1324 or other strains of adenovirus (e.g., Ad2, Ad3, Ad7 etc.) are well known to those skilled in the art. Adeno-associated virus (AAV) is a naturally occurring defective virus that requires another virus, such as an adenovirus or a herpes virus, as a helper virus for efficient replication and a productive life cycle. (For a review see Muzyczka et al., 1992, Curr. Topics in Micro. and Immunol. 158:97-129). An AAV vector such as that described in Traschin et al. (1985, Mol. Cell. Biol. 5:3251-3260) can be used to introduce nucleic acid into cells. A variety of nucleic acids have been introduced into different cell types using AAV vectors (see, for example, Hermonat et al., 1984, Proc. Natl. Acad. Sci. USA 81: 6466-6470; and Traschin et al., 1985, Mol. Cell. Biol. 4: 2072-2081).

Finally, the introduction and expression of foreign genes is often desired in insect cells because high level expression may be obtained, the culture conditions are simple relative to mammalian cell culture, and the post-translational modifications made by insect cells closely resemble those made by mammalian cells. For the introduction of foreign DNA to insect cells, such as Drosophila S2 cells, infection with baculovirus vectors is widely used. Other insect vector systems include, for example, the expression plasmid pIZ/V5-His (InVitrogen) and other variants of the pIZ/V5 vectors encoding other tags and selectable markers. Insect cells are readily transfectable using lipofection reagents, and there are lipid-based transfection products specifically optimized for the transfection of insect cells (for example, from PanVera).

B. Host Cells Useful According to the Invention.

Any cell into which recombinant vectors carrying a R. reniformis luciferase and R. reniformis GFP fusion protein genes or variants thereof may be introduced and wherein the vectors are permitted to drive the expression of R. reniformis luciferase and R. reniformis GFP fusion protein sequences is useful according to the invention. That is, because of the wide variety of uses for the BRET assay of the invention, any cell in which R. reniformis luciferase and R. reniformis GFP fusion protein genes of the invention may be expressed and preferably detected is a suitable host. Vectors suitable for the introduction of R. reniformis luciferase and R. reniformis GFP fusion protein-encoding sequences in host cells from a variety of different organisms, both prokaryotic and eukaryotic, are described herein above or known to those skilled in the art.

Host cells may be prokaryotic, such as any of a number of bacterial strains, or may be eukaryotic, such as yeast or other fungal cells, insect or amphibian cells, or mammalian cells including, for example, rodent, simian or human cells. Cells expressing R. reniformis luciferase and R. reniformis GFP fusion proteins of the invention may be primary cultured cells, for example, primary human fibroblasts or keratinocytes, or may be an established cell line, such as NIH3T3, 293T or CHO cells. Further, mammalian cells useful for expression of R. reniformis luciferase and R. reniformis GFP fusion proteins of the invention may be phenotypically normal or oncogenically transformed. It is assumed that one skilled in the art can readily establish and maintain a chosen host cell type in culture.

C. Introduction of R. reniformis Luciferase and R. reniformis GFP Fusion Protein-Encoding Vectors to Host Cells.

R. reniformis luciferase and R. reniformis GFP fusion protein-encoding vectors may be introduced to selected host cells by any of a number of suitable methods known to those skilled in the art. For example, R. reniformis luciferase and R. reniformis GFP fusion protein gene constructs may be introduced into appropriate bacterial cells by infection, in the case of E. coli bacteriophage vector particles such as lambda or M13, or by any of a number of transformation methods for compatible plasmid vectors or for bacteriophage DNA. For example, standard calcium-chloride-mediated bacterial transformation is still commonly used to introduce naked DNA to bacteria (Sambrook et al., 1989, Molecular Cloning, A Laboratory Manual, Cold Spring Harbor Laboratory Press, Cold Spring Harbor, N.Y.), but electroporation may also be used (Ausubel et al., 1988, Current Protocols in Molecular Biology, (John Wiley & Sons, Inc., NY, N.Y.)). For co-transformation of R. reniformis fusion genes into E. coli, two different compatible plasmid expression vectors need to be used each containing different antibiotic resistance genes.

For the introduction of R. reniformis luciferase and R. reniformis GFP fusion protein-encoding constructs into yeast or other fungal cells, chemical transformation methods are generally used (e.g. as described by Rose et al., 1990, Methods in Yeast Genetics, Cold Spring Harbor Laboratory Press, Cold Spring Harbor, N.Y.). For transformation of S. cerevisiae, for example, the cells are treated with lithium acetate to achieve transformation efficiencies of approximately 10⁴ colony-forming units (transformed cells)/μg of DNA. Transformed cells are then isolated on selective media appropriate to the selectable marker used. Alternatively, or in addition, plates or filters lifted from plates may be scanned for GFP fluorescence and luciferase-mediated bioluminescence to identify transformed clones with R. reniformis fusion protein gene constructs.

For the introduction of R. reniformis luciferase and R. reniformis GFP fusion protein-encoding vectors to mammalian cells, the method used will depend upon the form of the vector. For plasmid vectors, DNA encoding R. reniformis luciferase and R. reniformis GFP fusion protein sequences may be introduced by any of a number of transfection methods, including, for example, lipid-mediated transfection (“lipofection”), DEAE-dextran-mediated transfection, electroporation or calcium phosphate precipitation. These methods are detailed, for example, in Current Protocols in Molecular Biology (Fred M. Ausubel et al. (2001) John Wiley and Sons, Chapter 9)

Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, InVitrogen, JBL Scientific, MBI Fermentas, PanVera, Promega, Quantum Biotechnologies, Sigma-Aldrich, and Wako Chemicals USA.

For the introduction of R. reniformis luciferase and R. reniformis GFP fusion protein-encoding vectors to insect cells, such as Drosophila Schneider 2 cells (S2) cells, Sf9 or Sf21 cells, transfection is also performed by lipofection.

Following transfection with R. reniformis luciferase and R. reniformis GFP fusion protein-encoding vectors of the invention, eukaryotic (preferably, but not necessarily mammalian) cells successfully incorporating the construct (intra- or extrachromosomally) may be selected, as noted above, by either treatment of the transfected population with selection agents, such as an antibiotic whose resistance gene is encoded by one of the vectors, or by direct screening using, for example, FACS of the cell population or fluorescence and bioluminescence scanning of adherent cultures. Frequently, both types of screening may be used, wherein a negative selection is used to enrich for cells taking up the constructs and FACS or fluorescence and bioluminescence scanning are used to further enrich for cells expressing R. reniformis luciferase and R. reniformis GFP fusion proteins or to identify specific clones of cells, respectively. For example, negative selection with the neomycin analog G418 and hygromycin (Life Technologies, Inc.) may be used to identify cells that have received both a first recombinant vector encoding a R. reniformis luciferase fusion polypeptide and a hygromycin resistance gene and a second recombinant vector encoding a R. reniformis GFP fusion protein and a neomycin resistance gene. Bioluminescence emitted by luciferase activity of the R. reniformis luciferase fusion protein in the presence of coelentrazine and fluorescence scanning for R. reniformis GFP fusion protein may be used to identify those cells or clones of cells that express both R. reniformis luciferase and R. reniformis GFP fusion proteins.

For long-term, high-yield production of recombinant fusion proteins, stable expression is preferred. Rather than using expression vectors which contain viral origins of replication, host cells can be transformed with the R. reniformis fusion protein genes controlled by-appropriate expression control elements (e.g., promoter, enhancer, sequences, transcription terminators, polyadenylation sites, etc.), and selectable markers. The selectable marker in the recombinant plasmid confers resistance to the selection and allows cells to stably integrate the plasmid into their chromosomes. Commonly used selectable markers include neo, which confers resistance to the aminoglycoside G-418 (Colberre-Garapin, et al., J. Mol. Biol., 150:1, 1981); and hygro, which confers resistance to hygromycin (Santerre, et al., Gene, 30: 147, 1984). For example, R. reniformis luciferase fusion protein gene is cloned into an expression vector with a neomycin resistance gene whereas the R. reniformis GFP fusion protein gene is cloned into an expression vector containing a hygromycin resistance gene. Following transfection of both these recombinant expression vectors, cells are allowed to grow for 1-2 days in an enriched media, before being placed in a selective media containing both neomycin and hygromycin. After approximately 10 days, depending on the cell type used, the surviving neomycin and hygromycin resistant cells grow to form foci which in turn can be cloned and expanded into cell lines. In this manner, transfected cells contain integrated copies of both R. reniformis luciferase and GFP fusion protein genes.

A number of other selection systems may also be used, including but not limited to the herpes simplex virus thymidine kinase (Wigler, et al., Cell, 11: 223, 1977), hypoxanthine-guanine phosphoribosyltransferase (Szybalska & Szybalski, Proc. Nati. Acad. Sci. USA, 48:2026, 1962), and adenine phosphoribosyltransferase (Lowy, et al., Cell, 22: 817, 1980) genes, (in tk.sup.-, hgprt.sup.- or aprt cells respectively). Also, antimetabolite resistance can be used as the basis of selection for dhfr, which confers resistance to methotrexate (Wigler, et al., Proc. Natl. Acad. Sci. USA, 77: 3567, 1980; O'Hare, et al., Proc. Natl. Acad. Sci. USA, 8: 1527, 1981); gpt, which confers resistance to mycophenolic acid (Mulligan & Berg, Proc. Natl. Acad. Sci. USA, 78: 2072, 1981). Recently, additional selectable genes have been described, namely trpB, which allows cells to utilize indole in place of tryptophan; hisD, which allows cells to utilize histinol in place of histidine (Hartman & Mulligan, Proc. Natl. Acad. Sci. USA, 85:8047, 1988); and ODC (ornithine decarboxylase) which confers resistance to the ornithine decarboxylase inhibitor, 2-(difluoromethyl)-DL-ornithine, DFMO (McConlogue L., In: Current Communications in Molecular Biology, Cold Spring Harbor Laboratory, ed., 1987).

D. Expression of R. reniformis Luciferase and R. reniformis GFP Fusion Protein Genes in Transgenic Animals

Transgenic mice provide a useful tool for genetic and developmental biology studies and for the determination of the protein:protein interactions within a living organism. According to the method of conventional transgenesis, additional copies of normal or modified genes are injected into the male pronucleus of the zygote and become integrated into the genomic DNA of the recipient mouse. The transgene is transmitted in a Mendelian manner in established transgenic strains. Constructs useful for creating transgenic animals comprise genes under the control of either their normal promoters or an inducible promoter, reporter genes under the control of promoters to be analyzed with respect to their patterns of tissue expression and regulation, and constructs containing dominant mutations, mutant promoters, and artificial fusion genes to be studied with regard to their specific developmental outcome.

Typically, DNA fragments on the order of 10 kilobases or less are used to construct a transgenic animal (Reeves, 1998, New. Anat., 253:19). According to the invention, transgenic animals are created that harbor either a construct comprising a R. reniformis luciferase fusion protein fusion gene or R. reniformis GFP protein fusion gene according to the invention. Subsequent mating of R. reniformis luciferase fusion gene transgenic animals with R. reniformis GFP fusion gene transgenic animals results in 25% of the offspring harboring both R. reniformis luciferase and R. reniformis GFP fusion protein-encoding expression vectors. BRET assays can then be used to study protein:protein interaction between R. reniformis luciferase and R. reniformis GFP fusion proteins in cells from any tissue of the transgenic animal.

As used herein, the term “transgenic animal” refers to any animal, preferably a non-human mammal, bird, fish or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extra-chromosomally replicating DNA. In the typical transgenic animals described herein, the transgene causes cells to express a recombinant form of one of the subject polypeptide, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

Other Transgenic Animals

The invention provides for transgenic animals that include but are not limited to transgenic mice, rabbits, rats, pigs, sheep, horses, cows, goats, etc. A protocol for the production of a transgenic pig can be found in White and Yannoutsos, Current Topics in Complement Research: 64^{th} Forum in Immunology, pp. 88-94; U.S. Pat. No. 5,523,226; U.S. Pat. No. 5,573,933: PCT Application WO93/25071; and PCT Application WO95/04744. A protocol for the production of a transgenic mouse can be found in U.S. Pat. No. 5,530,177. A protocol for the production of a transgenic rat can be found in Bader and Ganten, Clinical and Experimental Pharmacology and Physiology, Supp. 3:S81-S87, 1996. A protocol for the production of a transgenic cow can be found in Transgenic Animal Technology, A Handbook, 1994, ed., Carl A. Pinkert, Academic Press, Inc. A protocol for the production of a transgenic rabbit can be found in Hammer et al., Nature 315:680-683, 1985 and Taylor and Fan, Frontiers in Bioscience 2:d298-308, 1997.

III. Expression and Purification of Fusion Proteins Comprising a Polypeptide Domain and R. reniformis GFP

In order to express a biologically active protein, the nucleotide sequence encoding the protein of interest or its functional equivalent, is inserted into an appropriate expression vector, i.e., a vector which contains the necessary elements for the transcription and translation of the inserted coding sequence.

Methods which are well known to those skilled in the art can be used to construct expression vectors containing a protein-encoding sequence and appropriate transcriptional or translational controls. These methods include in vivo recombination or genetic recombination. Such techniques are described in Ausubel et al., supra and Sambrook et al., supra.

A variety of expression vector/host systems may be utilized to contain and express a protein product of a candidate gene according to the invention. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus9 ); plant cell systems transfected with virus expression vector (e.g., cauliflower mosaic virus, CaMV; tobacco mosaic virus, TMV) or transformed with bacterial expression vectors (e.g., Ti or pBR322 plasmid); or animal cell systems.

The “control elements” or “regulatory sequences” of these systems vary in their strength and specificities and are those nontranslated regions of the vector, enhancers, promoters, and 3′ untranslated regions, which interact with host cellular proteins to carry out transcription and translation. Depending on the vector system and host utilized, any number of suitable transcription and translation elements, including constitutive and inducible promoters, may be used. For example, when cloning in bacterial systems, inducible promoters such as the hybrid lacZ promoter of the Bluescript® phagemid (Stratagene, LaJolla Calif.) or pSport1 (Gibco BRL) and ptrp-lac hybrids and the like may be used. The baculovirus polyhedron promoter may be used in insect cells. Promoters or enhancers derived from the genomes of plant cells (e.g., heat shock, RUBISCO; and storage protein genes) or from plant virus (e.g. viral promoters or leader sequences) may be cloned into the vector. In mammalian cell systems promoters from the mammalian genes or from mammalian viruses are most appropriate. If it is necessary to generate a cell line that contains multiple copies of the sequence encoding the protein product of the gene of interest, vectors based on SV40 or EBV may be used with an appropriate selectable marker.

In bacterial systems, a number of expression vectors may be selected depending upon the use intended for the protein of interest. For example, when large quantities of a protein are required for the production of antibodies, vectors which direct high level expression of fusion proteins that are readily purified may be desirable. Such vectors include, but are not limited to, the multifunctional E. coli cloning and expression vectors such as Bluescript® (Stratagene), in which the sequence encoding the protein of interest may be ligated into the vector in frame with sequences encoding the amino-terminal Met and the subsequent 27 residues of B-galactosidase so that a hybrid protein is produced; pIN vectors (Van Heeke & Schuster, 1989, J Biol Chem 264:5503); and the like. Pgex vectors (Promega, Madison Wis.) may also be used to express foreign polypeptides as fusion proteins with GST. In general, such fusion proteins are soluble and can easily be purified from lysed cells by adsorption to glutathione-agarose beads followed by elution in the presence of free glutathione. Proteins made in such systems are designed to include heparin, thrombin or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase and PGH may be used. For reviews, see Ausubel et al (supra) and Grant et al., 1987, Methods in Enzymology 153:516.

In cases where plant expression vectors are used, the expression of a sequence encoding a protein of interest may be driven by any of a number of promoters. For example, viral promoters such as the 35S and 19S promoters of CaMV (Brisson et al., 1984, Nature 310:511) may be used alone or in combination with the omega leader sequence from TMV (Takamatsu et al., 1987, EMBO J 6:307). Alternatively, plant promoters such as the small subunit of RUBISCO (Coruzzi et al., 1984, EMBO J 3:1671; Broglie et al., 1984, Science, 224:838); or heat shock promoters (Winter J and Sinibaldi R M, 1991, Results Probl Cell Differ., 17:85) may be used. These constructs can be introduced into plant cells by direct DNA transformation or pathogen-mediated transection. For reviews of such techniques, see Hobbs S or Murry L E in McGraw Hill Yearbook of Science and Technology (1992) McGraw Hill New York N.Y., pp 191-196 or Weissbach and Weissbach (1988) Methods for Plant Molecular Biology, Academic Press, New York, pp 421-463.

An alternative expression system which could be used to express a protein of interest is an insect system. In one such system, Autographa californica nuclear polyhedrosis virus (AcNPV) is used as a vector to express foreign genes in Spodoptera frugiperda cells or in Trichoplusia larvae. The sequence encoding the protein of interest may be cloned into a nonessential region of the virus, such as the polyhedrin gene, and placed under control of the polyhedrin promoter. Successful insertion of the sequence encoding the protein of interest will render the polyhedron gene inactive and produce recombinant virus lacking coat protein coat. The recombinant viruses are then used to infect S. frigoerda cells or Trichoplusia larvae in which the protein of interest is expressed (Smith et al., 1983., J. Virol 46:584; Engelhard, et al., 1994, Proc Nat Acad Sci 91:3224).

In mammalian host cells, a number of viral-based expression systems may be utilized. In cases where an adenovirus is used as an expression vector, a sequence encoding the protein of interest may be ligated into an adenovirus transcription/translation complex consisting of the late promoter and tripartite leader sequence. Insertion in a nonessential E1 or E3 region of the viral genome will result in a viable virus capable of expressing in infected host cells (Logan and Shenk, 1984, Proc Natl Acad Sci, 81:3655). In addition, transcription enhancers, such as the rous sarcoma virus (RSV) enhancer, may be used to increase expression in mammalian host cells.

Specific initiation signals may also be required for efficient translation of a sequence encoding the protein of interest. These signals include the ATG initiation codon and adjacent sequences. In cases where the sequence encoding the protein, its initiation codon and upstream sequences are inserted into the most appropriate expression vector, no additional translational control signals may be needed. However, in cases where only coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use (Scharf, et al., 1994, Results Probl Cell Differ, 20:125; Bittner et al., 1987, Methods in Enzymol, 153:516).

In addition, a host cell strain may be chosen for its ability to modulate the expression of the inserted sequences or to process the expressed protein in the desired fashion. Such modifications of the polypeptide include but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, etc have specific cellular machinery and characteristic mechanisms for such post-translational activities and may be chosen to ensure the correct modification and processing of the introduced, foreign protein.

For long-term, high-yield production of recombinant proteins, stable expression is preferred. For example, cell lines which stably express a foreign protein may be transformed using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clumps of stably transformed cells can be expanded using tissue culture techniques appropriate to the cell type.

Any number of selection systems may be used to recover transformed cell lines. These include, but are not limited to, the herpes simplex virus thymidine kinase (Wigler., et al., 1977, Cell 11:223) and adenine phosphoribosyltransferase (Lowy, et al., 1980, Cell 22:817) genes which can be employed in tk- or aprt-cells, respectively. Also, antimetabolite, antibiotic or herbicide resistance can be used as the basis for selection; for example, dhfr which confers resistance to methotrexate (Wigler et al., 1980, Proc Natl Acad Sci 77:3567); npt, which confers resistance to the aminoglycosides neomycin and G-418 (Colbere-Garapin et al., 1981., J Mol Biol., 150:1) and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman and Mulligan, 1988, Proc Natl Acad Sci 85:8047). Recently, the use of visible markers has gained popularity with such markers as anthocyanins, B glucuronidase and its substrate, GUS, and luciferase and its substrate, luciferin, being widely used not only to identify transformants, but also to quantify the amount of transient or stable protein expression attributable to a specific vector system (Rhodes et al., 1995, Methods Mol Biol 55:121).

IV. Preparation of Antibodies Reactive with Humanized R. reniformis GFP

Antibodies that bind to a GFP polypeptide encoded by a polynucleotide of the invention are useful, for example, in protein purification and in protein association assays. An antibody useful in the invention may comprise a whole antibody, an antibody fragment, a polyfunctional antibody aggregate, or in general a substance comprising one or more specific binding sites from an antibody. The antibody fragment may be a fragment such as an Fv, Fab or F(ab′)₂ fragment or a derivative thereof, such as a single chain Fv fragment. The antibody or antibody fragment may be non-recombinant, recombinant or humanized. The antibody may be of an immunoglobulin isotype, e.g., IgG, IgM, and so forth. In addition, an aggregate, polymer, derivative and conjugate of an immunoglobulin or a fragment thereof can be used where appropriate.

GFP-derived peptides used to induce specific antibodies preferably have an amino acid sequence consisting of at least five amino acids and more conveniently at least ten amino acids. It is advantageous for such peptides to be identical to a region of the natural R. reniformis GFP protein, and they may even contain the entire amino acid sequence of wild-type or humanized R. reniformis GFP (e.g., SEQ ID NO: 2).

For the production of antibodies, various hosts including goats, rabbits, rats, mice, etc., may be immunized by injection with peptides or polypeptides having sequences derived from the GFP polypeptides of the invention. Depending on the host species, various adjuvants may be used to increase the immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol.

To generate polyclonal antibodies, the antigen (i.e., an R. reniformis GFP polypeptide, or peptide fragment derived therefrom) may be conjugated to a conventional carrier in order to increase its immunogenicity, and an antiserum to the peptide-carrier conjugate raised. Short stretches of amino acids corresponding to a GFP polypeptide of the invention may be fused, either by expression as a fusion product or by chemical linkage, with amino acids from another protein such as keyhole limpet hemocyanin or GST, with antibodies then being raised against the chimeric molecule. Coupling of a peptide to a carrier protein and immunizations may be performed as described in Dymecki et al., 1992, J. Biol. Chem., 267:4815. The serum can be titered against polypeptide antigen by ELISA or alternatively by dot or spot blotting (Boersma & Van Leeuwen, 1994, J. Neurosci. Methods, 51:317). A useful serum will react strongly with the appropriate peptides by ELISA, for example, following the procedures of Green et al., 1982, Cell, 28:477.

Techniques for preparing monoclonal antibodies are well known, and monoclonal antibodies may be prepared using an antigen, preferably bound to a carrier, as described by Arnheiter et al., 1981, Nature, 294:278. Monoclonal antibodies are typically obtained from hybridoma tissue cultures or from ascites fluid obtained from animals into which the hybridoma tissue was introduced. Monoclonal antibody-producing hybridomas (or polyclonal sera) can be screened for antibody binding to the target protein according to methods known in the art.

V. Detecting Fluorescent Emission

FACS

Cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS method can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS machines collect fluorescence signals in one to several channels corresponding to different laser excitation and fluorescence emission wavelengths. Fluorescent labeling allows the investigation of many aspects of cell structure and function. The most widely used application is immunofluorescence: the staining of cells with antibodies conjugated to fluorescent dyes such as fluorescein and phycoerythrin. This method is often used to label molecules on the cell surface, but antibodies can also be directed at targets within the cell. In direct immunofluorescence, an antibody to a particular molecule, is directly conjugated to a fluorescent dye. Cells can then be stained in one step. In indirect immunofluorescence, the primary antibody is not labeled, but a second fluorescently conjugated antibody is added which is specific for the first antibody: for example, if the first antibody is a mouse IgG, then the second antibody could be a rat or rabbit antibody raised against mouse IgG.

FACS can also be used to measure BRET induced fluorescence.

FACS can be performed using an appropriate instrument, for example, a Becton-Dickinson FACSCalibur and CellQuest software.

Fluorescent Microscopy

BRET induced fluorescence can also be detected by fluorescence microscopy by methods described in Vives et al. (1997), J Biol Chem 272, 16010-7).

VI. Candidate Modulators Useful According to the Invention

The invention provides for a compound that is a modulator of a protein:protein interaction of the invention.

The candidate compound may be a synthetic compound, or a mixture of compounds, or may be a natural product (e.g. a plant extract or culture supernatant). A candidate compound according to the invention includes a small molecule that can be synthesized, a natural extract, peptides, proteins, carbohydrates, lipids etc . . . .

Candidate modulator compounds from large libraries of synthetic or natural compounds can be screened. Numerous means are currently used for random and directed synthesis of saccharide, peptide, and nucleic acid based compounds. Synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Combinatorial libraries are available and can be prepared. Alternatively, libraries of natural compounds in the form of bacterial, fungal, plant and animal extracts are available from e.g., Pan Laboratories (Bothell, Wash.) or MycoSearch (N.C.), or are readily produceable by methods well known in the art. Additionally, natural and synthetically produced libraries and compounds are readily modified through conventional chemical, physical, and biochemical means.

Useful compounds may be found within numerous chemical classes. Useful compounds may be organic compounds, or small organic compounds. Small organic compounds have a molecular weight of more than 50 yet less than about 2,500 daltons, preferably less than about 750, more preferably less than about 350 daltons. Exemplary classes include heterocycles, peptides, saccharides, steroids, and the like. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like.

For primary screening, a useful concentration of a candidate compound according to the invention is from about 1 μM to about 60 μM or more (i.e., 100 μM, 1 mM, 10 mM, 100 mM, 1M etc. . . ). The primary screening concentration will be used as an upper limit, along with nine additional concentrations, wherein the additional concentrations are determined by reducing the primary screening concentration at half-log intervals (e.g. for 9 more concentrations) for secondary screens or for generating concentration curves.

VII. Microarrays

A. Microarrays

Any combination of the fusion proteins of the invention are used for the construction of a microarray. A microarray according to the invention preferably comprises between 10 and 20,000 fusion proteins, and more preferably comprises at least 5000 fusion proteins.

In one embodiment, the above microarrays are used to identify a modulator that modulates a protein:protein interaction of the invention.

B. Construction of a Microarray

In one aspect, fusion proteins of the invention, for example fusion proteins which include a first R. reniformis GFP fusion polypeptide and a second R. reniformis luciferase fusion polypeptide are arrayed on a microarray.

In the subject methods, an array of fusion proteins comprising a first polypeptide domain fused toa R. reniformis GFP stably associated with the surface of a substantially planar solid support is contacted with a sample comprising fusion proteins comprising a second polypeptide fused to a R. reniformis luciferase under conditions sufficient to produce a binding pattern of first and second cognate polypeptide binding domains.

In another embodiment, an array comprising fusion proteins wherein each fusion protein at each position of the array comprises a unique first polypeptide domain fused to a R. reniformis GFP is contacted with a sample comprising a fusion protein, wherein the fusion protein comprises a second polypeptide domain fused to a R. reniformis luciferase. This array is used to identify binding partners of the second polypeptide domain. The identity of first polypeptide binding domains which bind to the second polypeptide domain can be determined with reference to the location of fusion proteins on the array.

A microarray according to the invention comprises a plurality of identical or unique fusion proteins attached to one surface of a solid support at a density exceeding 20 different fusion proteins/cm², wherein each of the fusion proteins is attached to the surface of the solid support in a non-identical pre-selected region. Each associated sample on the array comprises a fusion protein, of known identity, as described herein.

In the arrays of the invention, the fusion proteins are stably associated with the surface of a solid support, wherein the support may be a flexible or rigid solid support. By “stably associated” is meant that each fusion protein maintains a unique position relative to the solid support under hybridization and washing conditions. As such, the samples are non-covalently or covalently stably associated with the support surface. Examples of non-covalent association include non-specific adsorption, binding based on electrostatic interactions (e.g., ion pair interactions), hydrophobic interactions, hydrogen bonding interactions, specific binding through a specific binding pair member covalently attached to the support surface, and the like. Examples of covalent binding include covalent bonds formed between the fusion proteins and a functional group present on the surface of the rigid support (e.g., —OH), where the functional group may be naturally occurring or present as a member of an introduced linking group, as described in greater detail below

The amount of fusion protein present in each composition will be sufficient to provide for adequate binding and detection of target fusion protein during the assay in which the array is employed. Generally, the amount of each fusion protein stably associated with the solid support of the array is at least about 0.1 ng, preferably at least about 0.5 ng and more preferably at least about 1 ng, where the amount may be as high as 1000 ng or higher, but will usually not exceed about 20 ng. Where the nucleic acid member is “spotted” onto the solid support in a spot comprising an overall circular dimension, the diameter of the “spot” will generally range from about 10 to 5,000 μm, usually from about 20 to 2,000 μm and more usually from about 50 to 1000 μm.

C. Solid Substrate

An array according to the invention comprises either a flexible or rigid substrate. A flexible substrate is capable of being bent, folded or similarly manipulated without breakage. Examples of solid materials which are flexible solid supports with respect to the present invention include membranes, e.g., nylon, flexible plastic films, and the like. By “rigid” is meant that the support is solid and does not readily bend, i.e., the support is not flexible. As such, the rigid substrates of the subject arrays are sufficient to provide physical support and structure to the associated fusion proteins present thereon under the assay conditions in which the array is employed, particularly under high throughput handling conditions.

The substrate may be biological, non-biological, organic, inorganic, or a combination of any of these, existing as particles, strands, precipitates, gels, sheets, tubing, spheres, beads, containers, capillaries, pads, slices, films, plates, slides, chips, etc. The substrate may have any convenient shape, such as a disc, square, sphere, circle, etc. The substrate is preferably flat or planar but may take on a variety of alternative surface configurations. The substrate may be a polymerized Langmuir Blodgett film, functionalized glass, Si, Ge, GaAs, GaP, SiO₂, SIN_{4,} modified silicon, or any one of a wide variety of gels or polymers such as (poly)tetrafluoroethylene, (poly)vinylidenedifluoride, polystyrene, polycarbonate, or combinations thereof. Other substrate materials will be readily apparent to those of skill in the art upon review of this disclosure.

In a preferred embodiment the substrate is flat glass or single-crystal silicon. According to some embodiments, the surface of the substrate is etched using well-known techniques to provide for desired surface features. For example, by way of formation of trenches, v-grooves, mesa structures, or the like, the synthesis regions may be more closely placed within the focus point of impinging light, be provided with reflective “mirror” structures for maximization of light collection from fluorescent sources, etc.

Surfaces on the solid substrate will usually, though not always, be composed of the same material as the substrate. Alternatively, the surface may be composed of any of a wide variety of materials, for example, polymers, plastics, resins, polysaccharides, silica or silica-based materials, carbon, metals, inorganic glasses, membranes, or any of the above-listed substrate materials. In some embodiments the surface may provide for the use of caged binding members which are attached firmly to the surface of the substrate. Preferably, the surface will contain reactive groups, which are carboxyl, amino, hydroxyl, or the like. Most preferably, the surface will be optically transparent and will have surface Si—OH functionalities, such as are found on silica surfaces.

The surface of the substrate is preferably provided with a layer of linker molecules, although it will be understood that the linker molecules are not required elements of the invention. The linker molecules are preferably of sufficient length to permit fusion proteins of the invention and on a substrate to bind to other fusion proteins and to interact freely with molecules exposed to the substrate, for example a candidate modulator of the invention.

Often, the substrate is a silicon or glass surface, (poly)tetrafluoroethylene, (poly)vinylidendifluoride, polystyrene, polycarbonate, a charged membrane, such as nylon 66 or nitrocellulose, or combinations thereof. In a preferred embodiment, the solid support is glass. Preferably, at least one surface of the substrate will be substantially flat. Preferably, the surface of the solid support will contain reactive groups, including, but not limited to, carboxyl, amino, hydroxyl, thiol, or the like. In one embodiment, the surface is optically transparent. In a preferred embodiment, the substrate is a poly-lysine coated slide or Gamma amino propyl silane-coated Corning Microarray Technology-GAPS.

Any solid support to which a fusion protein may be attached may be used in the invention. Examples of suitable solid support materials include, but are not limited to, silicates such as glass and silica gel, cellulose and nitrocellulose papers, nylon, polystyrene, polymethacrylate, latex, rubber, and fluorocarbon resins such as TEFLON™.

The solid support material may be used in a wide variety of shapes including, but not limited to slides and beads. Slides provide several functional advantages and thus are a preferred form of solid support. Due to their flat surface, probe and hybridization reagents are minimized using glass slides. Slides also enable the targeted application of reagents, are easy to keep at a constant temperature, are easy to wash and facilitate the direct visualization of protein immobilized on the solid support. Removal of protein immobilized on the solid support is also facilitated using slides.

The particular material selected as the solid support is not essential to the invention, as long as it provides the described function. Normally, those who make or use the invention will select the best commercially available material based upon the economics of cost and availability, the expected application requirements of the final product, and the demands of the overall manufacturing process.

E. Spotting Method

In one aspect, the invention provides for arrays wherein each fusion protein comprising the array is spotted onto a solid support.

Preferably, spotting is carried out using a robotic GMS 417 arrayer (Affymetrix, CA).

The boundaries of the spots on the microarray are marked with a diamond scriber (note that the spots become invisible after post-processing).

Alternatively, spotting may be carried out using contact printing technology as is known in the art.

VIII. Polypeptides and Nucleic Acid Sequences Useful According to the Invention

A. Polypeptides of Interest According to the Invention

A polypeptide of interest includes any polypeptide with a known binding partner or any polypeptide suspected of having a binding partner.

As used herein, “binding partner” refers to a polypeptide or fragment thereof (peptide) that binds to a binding domain.

As used herein, the term “associates” or binds”refers to a polypeptide and its binding partner having a binding constant sufficiently strong to permit detection of binding by measuring fluorescent emission, preferably BRET-induced fluorescent emission, or other detection means, which are in physical contact with each other and have a dissociation constant (Kd) of about 10 μM or lower.

As used herein, “binding domain” refers in a three-dimensional sense to the amino acid residues of a first polypeptide domain required for binding of the first polypeptide domain to a second polypeptide domain. The amino acids of a binding domain may be either contiguous or non-contiguous. A binding domain must include at least 1 amino acid, and may include 2 or more, preferably 4 or more, amino acids which are contiguous or non-contiguous, and are necessary for binding of a first polypeptide domain to a second polypeptide domain. A “binding domain” may include a full length protein.

Proteins useful according to the methods of the invention also include but are not limited to receptors, enzymes, ligands, regulatory factors, and structural proteins. Therapeutic proteins including nuclear proteins, cytoplasmic proteins, mitochondrial proteins, secreted proteins, plasmalemma-associated proteins, serum proteins, viral antigens and proteins, bacterial antigens, protozoal antigens and parasitic antigens are also useful according to the invention.

Therapeutic proteins useful according to the invention also include lipoproteins, glycoproteins, phosphoproteins. Proteins or polypeptides which can be expressed using the methods of the present invention include hormones, growth factors, neurotransmitters, enzymes, clotting factors, apolipoproteins, receptors, drugs, oncogenes, tumor antigens, tumor suppressors, structural proteins, viral antigens, parasitic antigens and bacterial antigens. Specific examples of these compounds include proinsulin (GenBank #E00011), growth hormone, dystrophin (GenBank #NM₁₃ 007124), androgen receptors, insulin-like growth factor I (GenBank #NM_{—}00875), insulin-like growth factor II (GenBank #X07868) insulin-like growth factor binding proteins, epidermal growth factor TGF-α(GenBank #E02925), TGF-β (GenBank #AW008981), PDGF (GenBank #NM_{—}002607), angiogenesis factors (acidic fibroblast growth factor (GenBank #E03043), basic fibroblast growth factor (GenBank #NM_{—}002006) and angiogenin (GenBank #M11567)), matrix proteins (Type IV collagen (GenBank #NM_{—}000495), Type VII collagen (GenBank #NM_{—}000094), laminin (GenBank #J03202), phenylalanine hydroxylase (GenBank #K03020), tyrosine hydroxylase (GenBank #X05290)), oncogenes (ras (GenBank #AF 22080), fos (GenBank #k00650), myc (GenBank #J00120), erb (GenBank #X03363), src (GenBank #AH002989), sis GenBank #M84453), jun (GenBank #J04111)), E6 or E7 transforming sequence, p53 protein (GenBank #AH007667), Rb gene product (GenBank #m19701), cytokine receptor, Il-1 (GenBank #m54933), IL-6 (GenBank #e04823), IL-8 (GenBank #119591), viral capsid protein, and proteins from viral, bacterial and parasitic organisms which can be used to induce an immunologic response, and other proteins of useful significance in the body.

The compounds which can be incorporated are only limited by the availability of the nucleic acid sequence for the protein or polypeptide to be incorporated. One skilled in the art will readily recognize that as more proteins and polypeptides become identified they can be integrated into the DNA constructs of the invention and used in BRET assays, according to the methods of the present invention.

B. Nucleotide Sequences Useful According to the Invention

A nucleotide sequence useful according to the invention comprises any nucleotide sequence encoding a protein with a known binding partner or a protein suspected of having a binding partner.

1. Genes Encoding Toxins

Examples of genes useful in the invention include those encoding such agents including but not limited to genes encoding diphtheria toxin, Pseudomonas exotoxin, cholera toxin, pertussis toxin, etc., as follows. Diphtheria toxin-IL2 fusions for inhibition of HIV-1 infection (Zhang et al., 192, Jour. Acquired Immune Deficiency Syndrome 5:1181); Diphtheria toxin A chain for inhibition of HIV viral production (Harrison et al., 1992, AIDS Res. Hum. Retro. 8:39 and Curel et al., 1993, Hum. Gene Ther. 4:71); Diphtheria toxin A chain-liposome complexes for suppression of bovine leukemia virus infection (Kakidani et al., 1993, Microbiol. Immunol. 37:713); Diphtheria Toxin A chain gene coupled with immunoglobulin enhancers and promoters for B-cell toxicity (Maxwell et al., Cancer Res., 1991, 51:4299); Tat- and Rev-activated expression of a diphtheria toxin A gene (Harrison, 1991, Hum. Gene Ther. 2:53); Diphtheria toxin-CD4 fusion for killing of HIV-infected cells (Auilo et al., 1992, Eur. Mol. Biol. Org. Jour. 11:575).

Other toxins which are useful according to the invention include but are not limited to the following. Conditionally toxic retroviruses are disclosed in Brady et al., 1994, Proc. Nat. Aca. Sci. 91:365 and in Caruso et al., 1992, Bone Marrow Transplant, 9:187. Toxins against EBV infection are disclosed in Harris et al., 1991, Cell. Immunol. 134:85, and against poliovirus in Rodriguez et al., 1992, Jour. Virol. 66:1971. Toxins against influenza virus are disclosed in Bron et al., 1994, Biochemistry 33:9110.

2. Genes Encoding Immunoactive Agents

Another agent useful according to the invention includes immunoactive agents, i.e., agents which combat viral infections or production by activating an immune response to the virus. Such agents include but are not limited to cytokines against viruses in general (Biron, 1994, Curr. Opin. Immunol. 6:530); soluble CD4 against SIV (Watanabe et al., 1991, Proc. Nat. Aca. Sci. 88:126); CD4-immunoglobulin fusions against HIV-1 and SIV (Langner et al., 1993, Arch. Virol. 130:157); CD4(81-92)-based peptide derivatives against HIV infection (Rausch et al., 1992, Biochem. Pharmacol. 43:1785); lympho-cytotoxic antibodies against HIV infection (Szabo et al., 1992, Acta. Virol. 38:392); IL-2 against HIV infection (Bell et al., 1992, Clin Exp. Immunol. 90:6); and anti-T cell receptor antibodies against viruses in general (Newell et al., 1991, Ann. N.Y. Aca. Sci. 636:279).

3. Genes Encoding Anti-Viral Drugs

Genes encoding anti-viral agents useful according to the invention include genes encoding drugs having anti-viral activity and which are the direct product of a gene or are a product of a gene encoding a precursor of the drug, the drug then being synthesized by a biosynthetic pathway in the cell. Targets of drug intervention in the replicative cycle of, for example, a retrovirus, include (1) binding and entry, (2) reverse transcriptase, (3) transcription and translation, and (4) viral maturation and budding. Representative inhibitors of viral binding and entry for HIV include recombinant soluble CD4, immunoadhesions, peptide T, and hypericin. Nucleoside reverse transcriptase inhibitors include zidovudine, didanosine, zalcitabine, and starudine. Foscarnet, tetrahydroimidazobenzodiazepinethione compounds, and nevirapine are some non-nucleoside reverse transcriptase inhibitors. Inhibitors of transcription and translation include antagonists of the TAT gene and GLQ223. Castanospermine and protease inhibitors interfere with viral budding and maturation. Such drugs include but are not limited to nucleoside or nucleotide analogs and products of a cellular biosynthetic pathway such as described in Harrell et al., 1994, Drug Metab. Dispos. 22:124 (deoxy-guanine); Fillon et al., 1993, Clin. Invest. Med. 16:339 (dauno-rubicin); Ohrvi et al., 1990, Nucleic Acids Symp. 26:93 (anti-viral nucleosides); Hudson et al., 1993, Photochem. Photobiol. 57:675 (thiarubines); Salhany et al., 1993, Jour. Biol. Chem. 268:7643 (pyridoxal 5′-phosphate); Damaso et al., 1994, Arch. Viral. 134:303 (cyclosporin A); Gallicchio et al., 1993. Int. Jour. Immunol. 15:263 (dideoxynucleoside drugs); and Fiore et al., 1990, Biol. Soc. Ital. Biol. Sper. 66:601 (AZT).

IX. The Use of the BRET Assay for the Study of Protein-Protein Interactions

A. In vivo Assays

1. Detection of Protein:Protein Interaction in Cells

The BRET system is useful for in vivo assays. In general, a first fusion protein comprising a first polypeptide domain and a R. reniformis-GFP and a second fusion protein comprising a second polypeptide domain and a R. reniformis luciferase will be introduced by transforming or transfecting a cell with one or more vectors comprising the recombinant nucleic acids encoding these fusion proteins. The cell will produce the fusion proteins and BRET will occur when the luciferase, the fluorophore and the substrate are in the appropriate spatial relationship.

2. Detection of Protein:Protein Interaction in Transgenic Animals

Transgenic animals comprising a first fusion protein comprising a first polypeptide domain and a R. reniformis GFP (encoded by a humanized polynucleotide sequence) and a second fusion protein comprising a second polypeptide domain and a R. reniformis luciferase are prepared as described herein. A protein:protein interaction between the first and second polypeptide domains is detected in the transgenic animal by performing fluorescent microscopy on the various organs of the transgenic animal or on tissue sections prepared from the transgenic animal. Alternatively, a cell type of interest is isolated from the transgenic animal and analyzed for fluorescent emission in an instrument capable of detecting BRET-induced fluorescence or by FACS.

3. Determination of the Subcellular Location of a Protein:Protein Interaction

The subcellular location of a protein:protein interaction is determined by performing fluorescence microscopy on cells transfected with a vector encoding a first fusion protein comprising a first polypeptide domain and a R. reniformis GFP (encoded by a humanized polynucleotide sequence) and a second fusion protein comprising a second polypeptide domain and a R. reniformis luciferase, as described herein.

B. In vitro Assays

The components of the BRET system (i.e. a first fusion protein comprising a first polypeptide domain and a R. reniformis GFP (encoded by a humanized polynucleotide sequence) and a second fusion protein comprising a second polypeptide domain and a R. reniformis) Luciferase can be produced using molecular biology techniques, as described herein, or isolated from natural sources. After purification, they can be used in non-cell based in vitro assays (as described in Example 1).

X. The Use of the BRET Assay for the Identification of Candidate Modulators of Protein-Protein Interactions

A. In vivo

A first fusion protein comprising a first polypeptide domain and a R. reniformis-GFP and a second fusion protein comprising a second polypeptide domain and a R. reniformis luciferase will be introduced by transforming or transfecting a cell with one or more vectors comprising the recombinant nucleic acids encoding these fusion proteins. The cell will produce the fusion proteins and BRET will occur when the luciferase, the fluorophore and the substrate are in the appropriate spatial relationship.

A replicate sample of cells is treated with a candidate modulator. BRET-induced fluorescence is detected and compared in the presence and absence of the modulator.

B. In vitro

The components of the BRET system (i.e. a first fusion protein comprising a first polypeptide domain and a R. reniformis GFP (encoded by a humanized polynucleotide sequence) and a second fusion protein comprising a second polypeptide domain and a R. reniformis) Luciferase can be produced using molecular biology techniques, as described herein, or isolated from natural sources. After purification, they can be used in non-cell based in vitro assays in the presence and absence of a candidate modulator of the invention.

C. Adaptability of BRET to Automation and High-Throughput Screening

The BRET system is adaptable to means of automation and high-throughput screening. A relatively simple scheme for designing an in vivo library screening system for protein-protein interaction using BRET is envisaged. By sensitively measuring the light emission collected through interference filters, the 510 nm/460-480 nm luminescence ratio of E. coli (or yeast) colonies expressing a “bait” protein fused to R. reniformis luciferase and a library of “prey” molecules fused to R. reniformis GFP (or vice versa) could be measured. Colomes that express an above-background ratio could be saved and the “prey” DNA sequence further characterized. Expression vectors would need the following features: multiple cloning sites for insertion of the bait and prey libraries to enable both N-terminal and C-terminal fusions of the bait molecule and prey library to R. reniformis luciferase and R. reniformis GFP, and an assortment of antibiotic or other markers to allow various expression/terminal fusion combinations to be tested. With appropriate instrumentation, high-throughput screening using BRET is a possibility. Using an imaging instrument similar, it is possible to screen colonies of bacteria or yeast on agar plates. On the other hand, a photomultiplier-based instrument designed to measure luminescence of liquid cultures in 96-well plates could be made ideal for high-throughput BRET screening by insertion of switchable 460-480 or 510 nm interference filters in front of the photomultiplier tube.

XI The Use of the BRET Assay as a Biosensor

The invention also provides for a biosensor molecule comprising R. reniformis hrGFP and R. reniformis luciferases that are fused together using a linker sequence. Upon addition of coelentrazine, BRET ensues because GFP and luciferase are in close proximity to each other. However, cleavage of the linker sequence effectively separates GFP and luciferase and results in a decrease in BRET. This simple assay can therefore be used to screen for inhibitors of an enzyme that cleaves the linker sequence between GFP and luciferase.

For example, the biosensor can be used to monitor apoptosis in vivo. According to this scenario, the linker is engineered to include a DEVD peptide, which is a caspase 3 substrate. With the initiation of apoptosis, caspase 3 is synthesized and cleaves DEVD sequences including the DEVD sequence within the GFP-DVED-R.Luc fusion protein. GFP and luciferase are then released and BRET decreases because GFP and Luc are no longer in close proximity to each other. For reference, see Angers S. et al.(2000):PNAS vol.97(7), 3684-89.

### EXAMPLES

The invention is illustrated by the following nonlimiting examples wherein the following materials and methods are employed. The entire disclosure of each of the literature references cited hereinafter are incorporated by reference herein.

### Example 1

This Example teaches how to use a hrGFP to detect a protein:protein interaction with a BRET assay.

A first fusion protein comprising a nucleotide sequence encoding the EGF receptor fused in frame to a humanized nucleotide sequence encoding a R. reniformis GFP polypeptide and a second fusion protein comprising a nucleotide sequence encoding EGF fused in frame to a nucleotide sequence encoding a R. reniformis luciferase polypeptide are produced by any of the methods described herein.

The first and second fusion proteins and a substrate for luciferase, for example coelenterazine, are mixed under conditions of salt and temperature that permit binding of the EGF receptor to EGF.

The fluorescent emission from the R. reniformis GFP polypeptide is detected by collecting readings using a modified topcount apparatus (BRETCount) that allows the sequential integration of the signals detected in the 440- to 500-nm and 510- to 590-nm windows (Angers et al., 2000, Proc. Natl. Acad Sci. USA, 97:3684-3689). Alternatively, the emission spectrum (400-600 nM) is immediately acquired using a Spex fluorolog spectrofluorimeter with the excitation lamp turned off. For comparisons between experiments, emission spectra are normalized with the peak emission from Renilla luciferase in the region of 480 nm being defined as an intensity of 1.00. In some cases a BRET signal is calculated by measuring the area under the curve between 500 and 550 nm. Background is taken as the area of this region of the spectrum when examining emission from the isolated Renilla luciferase (McVey et al., 2001, J. Biol. Chem., 276:14092-14099). In another embodiment, repeated readings are taken for at least 5-10 min using a custom designed BRET instrument (Berthold, Australia) which allows sequential integration of the signals detected in the 440-500 and 510-590 nm windows. Data are represented as a normalized BRET ratio, which is defined as the BRET ratio for the co-expression of the Rluc and hrGFP constructs normalized against the BRET ratio for the Rluc expression construct alone. The BRET ratio is defined as ((emission at 510-590 nm)−(emission at 440-500 nm)×cf)/(emission at 440-500 mn), where cf corresponds to (emission at 510-590 nm/emission at 440-500 nm) for the Rluc construct expressed alone in the same experiment (Kroeger et al., 2001, J. Biol. Chem., 276:12736-12743).

### Example 2

This Example teaches how to use a hrGFP to determine the location of a protein:protein interaction with a BRET assay.

A cell line useful according to the invention is transected with a first vector encoding a fusion protein comprising a nucleotide sequence encoding the EGF receptor fused in frame to a humanized nucleotide sequence encoding a R. reniformis GFP polypeptide. The first vector also comprises a neomycin resistance gene. The cells are also transfected with a second vector encoding a fusion protein comprising a nucleotide sequence encoding EGF fused in frame to a nucleotide sequence encoding a R. reniformis luciferase polypeptide. The second vector also comprises a hygromycin resistance gene.

Cells that have taken up both the first and second vector are selected by growth in medium containing both neomycin and hygromycin. Expression of the first and second fusion proteins is confirmed by Western blot analysis or immunoprecipitation, according to methods well-known in the art.

Cells that are expressing both the first and second fusion proteins are mixed with a substrate for luciferase, for example coelenterazine added to a concentration of 5-10 □M.

The fluorescent emission from the R. reniformis GFP polypeptide is detected as described in Example 1.

### Example 3

This Example teaches how to use a hrGFP to identify a modulator that increases or decreases a protein:protein interaction with a BRET assay in vivo.

A cell line useful according to the invention is transected with a first vector encoding a fusion protein comprising a nucleotide sequence encoding the EGF receptor fused in frame to a humanized nucleotide sequence encoding a R. reniformis GFP polypeptide. The first vector also comprises a neomycin resistance gene. The cells are also transfected with a second vector encoding a fusion protein comprising a nucleotide sequence encoding EGF fused in frame to a nucleotide sequence encoding a R. reniformis luciferase polypeptide. The second vector also comprises a hygromycin resistance gene.

Cells that have taken up both the first and second vector are selected by growth in medium containing both neomycin and hygromycin. Expression of the first and second fusion proteins is confirmed by Western blot analysis or immunoprecipitation, according to methods well-known in the art.

Cells that are expressing both the first and second fusion proteins are mixed with a substrate for luciferase, for example coelenterazine added to a concentration of 5-10 μM. Replicate samples of the cells are preincubated with a candidate modulator for various time points in the range of 5 min to 24 hours prior to the addition of coelenterazine.

The fluorescent emission from the R. reniformis GFP polypeptide from cells treated in the presence or absence of a candidate modulator is detected as described in Example 1.

The level of fluorescent emission from the R. reniformis GFP is compared in the presence or absence of a candidate modulator.

### Example 4

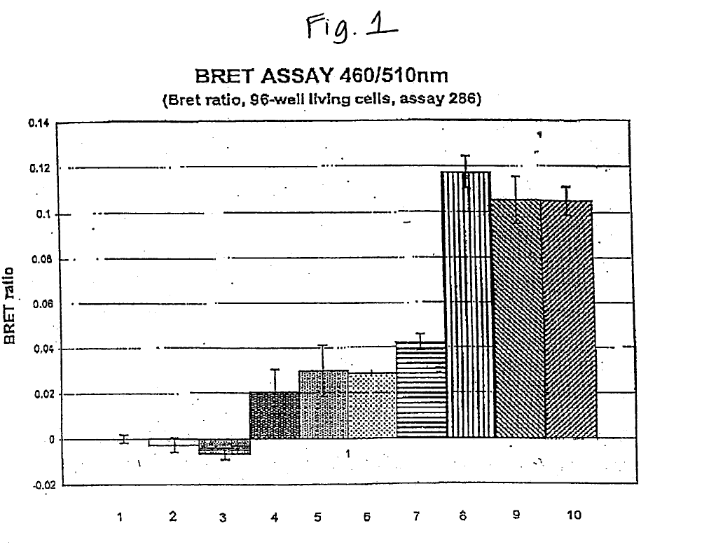

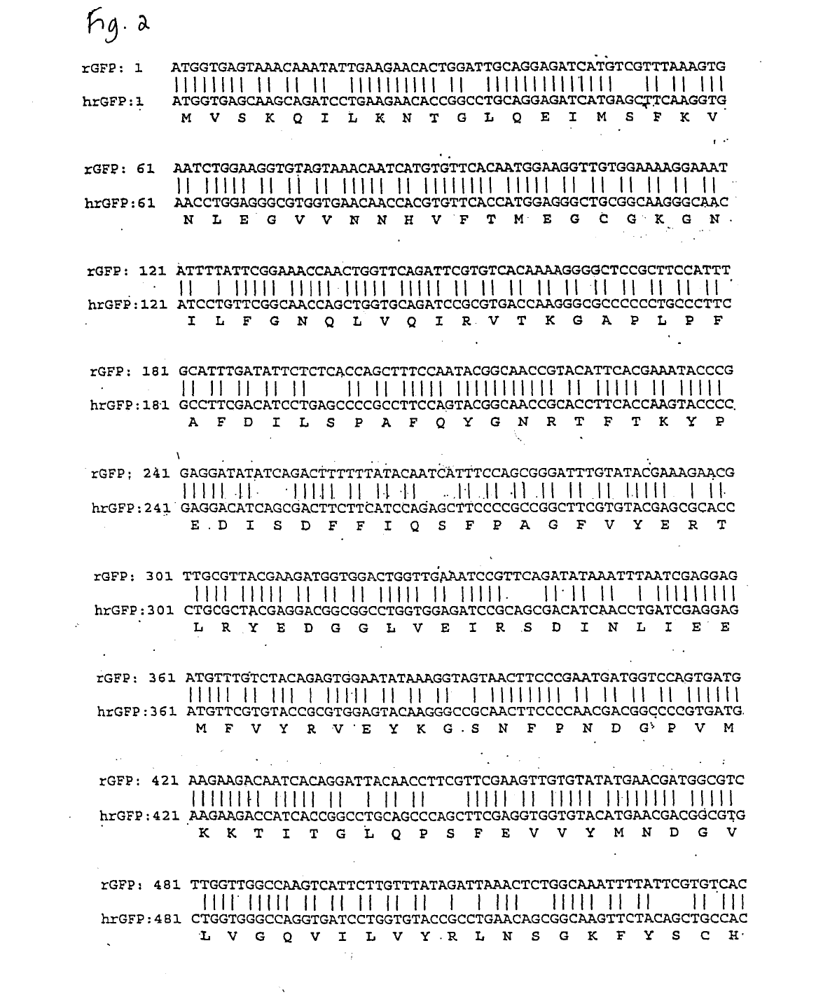

This Example describes a BRET assay of the invention (see FIG. 1)

293×cells were transfected with the following plasmids: Lane 1-pCMV-Rluc (luciferase only) (0.1 μg); Lane 2-pAAVRluclRES-hrGFP (0.1 μg); Lane 3-pAAVRluclRES-hrGFP (0.2 μg); Lane 4-pCMV-Rluc and pAAVhrGFP (two proteins translated separately) (0.1 μg); Lane 5-pCMV-Rluc and pAAVhrGFP (two proteins translated separately) (0.3 μg); Lane 6-pBRET+(positive control plasmid from Packard); Lane 7: pAAVRluc-hrGFP, Rluc and hrGFP fusion protein, Rluc is at N-terminal and hrGFP at C-terminal; Lane 8: pCMVhrGFP-Rluc clone#1, hrGFP and Rluc fusion protein; hrGFP is at N-terminal and Rluc at C-terminal; Lane 9: same as lane 8 except clone #5; Lane 10: same as lane 8 except clone#6. Cells were transiently transfected into each well of a 96-well plate by the Ca⁺⁺ method. Forty-eight hours later, the media were removed and the cells were incubated with 25 μl of 1×BRET buffer (1×PBS pH7.4, 1 mM CaCl2, 0.5mM MgCl2, 1 mg/ml D-glucose). 75 μl of coelenterazine in BRET buffer was added into each well to a final coelenterazine at a concentration of 2 μM. The plate was immediately read at emission 460 nm and 510 nm in a Victor2 plate reader (Perkin Elmer, Gaithersburg, Md.).

1. A composition comprising a first polynucleotide comprising and expression cassette containing a sequence encoding R. reniformis luciferase and a second polynucleotide comprising an expression cassette containing a sequence encoding R. reniformis GFP.

2. The composition of claim 1, wherein the polynucleotide sequence of R. reniformis GFP contains at least one codon which is humanized.

3. The composition of claim 1, further comprising a substrate for luciferase.

4. The composition of claim 3, wherein said substrate is coelentrazine.

5. The composition of claim 1, wherein said first and second polynucleotides are present in a single vector.

6. The composition of claim 1, wherein one or both of said expression cassettes contain multiple cloning sites.

7. The composition of claim 6, wherein each said multiple cloning site is at N terminus or C terminus of said sequence encoding R. reniformis luciferase and R. reniformis GFP respectively.

8. The composition of claim 1, further comprising a first coding region of interest inserted into said first polynucleotide expression cassette.

9. The composition of claim 1, further comprising a second coding region inserted into said second polynucleotide expression cassette.

10. A composition comprising a first recombinant fusion protein comprising a first polypeptide fused to the amino acid sequence of R. reniformis luciferase and a second recombinant fusion protein comprising a second polypeptide fused to the amino acid sequence of R. reniformis GFP.

11. The composition of claim 10, wherein the polynucleotide sequence encoding R. reniformis GFP polypeptide is humanized.

12. The composition of claim 10, wherein the polynucleotide sequence encoding R. reniformis GFP comprises the sequence of SEQ ID NO: 1.

13. The composition of claim 10, further comprising a substrate for luciferase.

14. The composition of claim 13, wherein the substrate is coelentrazine.

15. The composition of claim 1, further comprising packaging materials therefore.

16. A method of detecting protein: protein interactions, said method comprising:
a) contacting a first fusion protein comprising a first polypeptide fused to R. reniformis luciferase polypeptide, and a second fusion protein comprising a second polypeptide fused to R. reniformis GFP polypeptide and a substrate for luciferase under conditions sufficient to permit BRET;
b) detecting BRET induced fluorescent emission from said R. reniformis GFP, wherein said fluorescent emission from said R. reniformis GFP indicates protein:protein interaction between said first and said second polypeptides.

17. The method of claim 16, wherein said substrate is coelentrazine.

18. The method of claim 16, wherein said method is performed in a cell.

19. The method of claim 16, wherein said method is performed in a cell membrane comprising said first and second fusion proteins.

20. A method of determining the location in a cell of a protein:protein interaction between two polypeptides, said method comprising the steps of:
a) introducing into a cell a first polynucleotide sequence encoding a first polynucleotide comprising an expression cassette containing a sequence encoding R. reniformis luciferase and a second polynucleotide comprising an expression cassette containing a sequence encoding R. reniformis GFP;
b) contacting said cell of step a) with a substrate for luciferase;
c) detecting BRET induced fluorescent emission from said R. reniformis GFP as an indicator of the location of the cellular compartment where said protein:protein interaction between said first and said second polypeptides occurs.

21. The composition of claim 20, wherein the polynucleotide sequence of R. reniformis GFP contains at least one codon which is humanized.

22. The composition of claim 20, wherein said substrate is coelentrazine.

23. The composition of claim 20, wherein said first and second polynucleotides are present in a single vector.

24. The composition of claim 20 wherein one or both of said expression cassettes contain multiple cloning sites.

25. The composition of claim 20 wherein each said multiple cloning site is at N terminal or C terminal of said sequence encoding R. reniformis luciferase and R. reniformis GFP respectively;

26. The composition of claim 20 further comprising a first coding region of interest inserted into said first polynucleotide expression cassette;

27. The composition of claim 20 further comprising a second coding region inserted into said second polynucleotide expression cassette.

28. A method of identifying cells in which there is a protein:protein interaction between two polypeptides of interest, said method comprising the steps of:
a) introducing into a population of cells a first polynucleotide sequence encoding a first polynucleotide comprising an expression cassette containing a sequence encoding R. reniformis luciferase and a second polynucleotide comprising an expression cassette containing a sequence encoding R. reniformis GFP;
b) contacting said cell of step a) with a substrate for luciferase;
c) detecting BRET induced fluorescent emission from said R. reniformis GFP, wherein said fluorescent emission from said R. reniformis GFP identifies a cell in which a protein:protein interaction between said first and said second polypeptides occurs.

29. The composition of claim 28, wherein the polynucleotide sequence of R. reniformis GFP contains at least one codon which is humanized.

30. The composition of claim 28, wherein said substrate is coelentrazine.

31. The composition of claim 28, wherein said first and second polynucleotides are present in a single vector.

32. The composition of claim 28, wherein one or both of said expression cassettes contain multiple cloning sites.

33. The composition of claim 28 wherein each said multiple cloning site is at N terminal or C terminal of said sequence encoding R. reniformis luciferase and R. reniformis GFP respectively.

34. The composition of claim 28 further comprising a first coding region of interest inserted into said first polynucleotide expression cassette.

35. The composition of claim 28 further comprising a second coding region inserted into said second polynucleotide expression cassette.

36. The method of claims 20 or 28, wherein said detection involves fluorescent activated cell sorter (FACS) analysis.

37. The method of claim 28, wherein population of cells is a tissue obtained from a transgenic animal.

38. The method of claim 28, wherein said population of cells are transformed with a single polynucleotide sequence encoding both a first fusion protein comprising a first polypeptide domain and a R. reniformis GFP polypeptide, and a second fusion protein comprising a second polypeptide domain and a R. reniformis luciferase polypeptide.

39. A method of screening for a candidate modulator that modulates a protein:protein interaction between two polypeptides, said method comprising:
a) contacting a first fusion protein comprising a first polypeptide and R. reniformis GFP polypeptide, and a second fusion protein comprising a second polypeptide and R. reniformis luciferase polypeptide, a candidate modulator, under conditions that permit binding of said fusion polypeptides to each other, and a substrate for luciferase;
b) measuring BRET induced fluorescent emission, wherein said fluorescent emission indicates a protein:protein interaction between said first and said second polypeptides and;
c) comparing the amount of fluorescence emission in step b) to the amount of fluorescence emission in the absence of said candidate modulator.

40. The method of claim 39, wherein said substrate is coelentrazine.

41. The method of claim 39, wherein said method is performed in a cell.

42. The method of claim 39, wherein said first and second fusion proteins are present in a cell membrane.

43. The method of claim 39, wherein said candidate modulator is selected from the group consisting of a natural or synthetic peptide, a polypeptide, an antibody or antigen-binding fragment thereof, a lipid, a carbohydrate, a nucleic acid, and a small organic molecule.

44. The method of claim 39, wherein said step of measuring comprises detecting a change in the level of fluorescent emission from said R. reniformis GFP in the presence of a candidate modulator as compared to the absence of a candidate modulator.

45. The method of claim 39, wherein said method is performed in a microarray.

46. The method of claims 16, 20, 28 or 39 where said first polypeptide and said second polypeptide are identical.

47. The method of claims 16, 20, 28 or 39 wherein said first and second polypeptides are receptor domains or portions thereof.

48. A kit for detecting a protein:protein interaction, said kit comprising a first polynucleotide comprising an expression cassette containing a sequence encoding R. reniformis luciferase and a second polynucleotide comprising an expression cassette containing a sequence encoding R. reniformis GFP and packaging materials therefore wherein the first and second polynucleotides are packaged independently.

49. A kit for comprising a polynucleotide encoding a R. reniformis luciferase polypeptide and a R. reniformis GFP polypeptide, and packaging materials therefore.

50. The kit of claims 48 or 49 further comprising a substrate for luciferase.

51. The kit of claim 50, wherein the said substrate is coelentrazine.

52. The kit of claim 48, further comprising fluorescently labeled antibodies specific for different cellular compartments, wherein said kit is used to detect protein:protein interaction in said cellular compartments.

53. The kit of claim 48, further comprising a standard BRET system, wherein said kit is used to identify cells in which there is a protein:protein interaction.

54. The kit of claim 48, further comprising a modulator of a standard BRET system, wherein said kit is used for screening for agents that modulate a protein:protein interaction.


SEQUENCE LISTING (30 sequences)

SEQ ID NO: 1 - DNA, 720 bp, Renilla reniformis
  exon (1)..(720): open reading frame
atggtgagtaaacaaatattgaagaacactggattgcaggagatcatgtcgtttaaagtgaatctggaaggtgtagtaaacaatcatgtgttcacaatggaaggttgtggaaaaggaaatattttattcggaaaccaactggttcagattcgtgtcacaaaaggggtcccgcttccatttgcatttgatattctctcaccagctttccaatacggcaaccgtacattcacgaaatacccggaggatatatcagacttttttatacaatcatttccagcgggatttgtatacgaaagaacgttgcgttacgaagatggtggactggttgaaatccgttcagatataaatttaatcgaggagatgtttgtctacagagtggaatataaaggtagtaacttcccgaatgatggtccagtgatgaagaagacaatcacaggattacaaccttcgttcgaagttgtgtatatgaacgatggcgtcttggttggccaagtcattcttgtttatagattaaactctggcaaattttattcgtgtcacatgagaacactgatgaaatcaaagggtgtagtgaaggattttcccgaataccatttcattcaacatcgtttagagaagactgatgtggaagacggaggttttgttgagcaacacgagacggccattgctcaactgacatcgctggggaaaccacttggatccttacacgaatgggtttaa

SEQ ID NO: 2 - protein, 239 aa, Renilla reniformis
MVSKQILKNTGLQEIMSFKVNLEGVVNNHVFTMEGCGKGNILFGNQLVQIRVTKGVPLPFAFDILSPAFQYGNRTFTKYPEDISDFFIQSFPAGFVYERTLRYEDGGLVEIRSDINLIEEMFVYRVEYKGSNFPNDGPVMKKTITGLQPSFEVVYMNDGVLVGQVILVYRLNSGKFYSCHMRTLMKSKGVVKDFPEYHFIQHRLEKTDVEDGGFVEQHETAIAQLTSLGKPLGSLHEWV

SEQ ID NO: 3 - DNA, 720 bp, Artificial sequence
  Humanized DNA
atggtgagcaagcagatcctgaagaacaccggcctgcaggagatcatgagcttcaaggtgaacctggagggcgtggtgaacaaccacgtgttcaccatggagggctgcggcaagggcaacatcctgttcggcaaccagctggtgcagatccgcgtgaccaagggcgcccccctgcccttcgccttcgacatcctgagccccgccttccagtacggcaaccgcaccttcaccaagtaccccgaggacatcagcgacttcttcatccagagcttccccgccggcttcgtgtacgagcgcaccctgcgctacgaggacggcggcctggtggagatccgcagcgacatcaacctgatcgaggagatgttcgtgtaccgcgtggagtacaagggccgcaacttccccaacgacggccccgtgatgaagaagaccatcaccggcctgcagcccagcttcgaggtggtgtacatgaacgacggcgtgctggtgggccaggtgatcctggtgtaccgcctgaacagcggcaagttctacagctgccacatgcgcaccctgatgaagagcaagggcgtggtgaaggacttccccgagtaccacttcatccagcaccgcctggagaagacctacgtggaggacggcggcttcgtggagcagcacgagaccgccatcgcccagctgaccagcctgggcaagcccctgggcagcctgcacgagtgggtgtaa

SEQ ID NO: 4 - DNA, 4079 bp, artificial sequence
  phRL- CMV plasmid sequence
agatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 5 - DNA, 44 bp, Artificial
  Synthetic primer
aattattagaattcaccatggtgagtaaacaaatattgaagaac

SEQ ID NO: 6 - DNA, 38 bp, Artificial
  Synthetic primer
ataatattctcgagttaaacccattcgtgtaaggatcc

SEQ ID NO: 7 - DNA, 12 bp, artificial sequence
  DNA codons for human use
gccgctgcagcg

SEQ ID NO: 8 - DNA, 6 bp, artificial sequence
  DNA codons for human use
tgttgt

SEQ ID NO: 9 - DNA, 6 bp, artificial sequence
  DNA codons for human use
gacgat

SEQ ID NO: 10 - DNA, 6 bp, artificial sequence
  DNA codons for human use
gaggaa

SEQ ID NO: 11 - DNA, 6 bp, artificial sequence
  DNA codons for human use
ttcttt

SEQ ID NO: 12 - DNA, 12 bp, artificial sequence
  DNA codons for human use
ggcgggggaggt

SEQ ID NO: 13 - DNA, 6 bp, artificial sequence
  DNA codons for human use
caccat

SEQ ID NO: 14 - DNA, 9 bp, artificial sequence
  DNA codons for human use
atcattata

SEQ ID NO: 15 - DNA, 6 bp, artificial sequence
  DNA codons for human use
aagaaa

SEQ ID NO: 16 - DNA, 15 bp, artificial sequence
  DNA codons for human use
ctgttgcttctatta

SEQ ID NO: 17 - DNA, 3 bp, artificial sequence
  DNA codon for human use
atg

SEQ ID NO: 18 - DNA, 6 bp, artificial sequence
  DNA codons for human use
aacaat

SEQ ID NO: 19 - DNA, 12 bp, artificial sequence
  DNA codons for human use
ccccctccaccg

SEQ ID NO: 20 - DNA, 6 bp, artificial sequence
  DNA codons for human use
cagcaa

SEQ ID NO: 21 - DNA, 18 bp, artificial sequence
  DNA codons for human use
cgcaggcggagacgacgt

SEQ ID NO: 22 - DNA, 18 bp, artificial sequence
  DNA codons for human use
agctcctctagttcatcg

SEQ ID NO: 23 - DNA, 12 bp, artificial sequence
  DNA codons for human use
accacaactacg

SEQ ID NO: 24 - DNA, 12 bp, artificial sequence
  DNA codons for human use
gtggtcgttgta

SEQ ID NO: 25 - DNA, 3 bp, artificial sequence
  DNA codon for human use
tgg

SEQ ID NO: 26 - DNA, 6 bp, artificial sequence
  DNA codons for human use
tactat

SEQ ID NO: 27 - DNA, 102 bp, artificial sequence
  Multiple Cloning Site Region of phrGFP-N1
ctgcacgagtgggtggagctctccggactcagatctcgagctgaagcttcgaatgcagtcgacggtaccgcgggcccgggatccaccggatctagataatag

SEQ ID NO: 28 - DNA, 116 bp, artificial sequence
  Multiple Cloning Site Region of phrGFP-C
agctggagctccaccgcggtggcggccgctctagcccgggcggatcccccgggctgcaggaattcgatatcaagcttatcgataccgtcgacctcgagaccatggtgagcaagcag

SEQ ID NO: 29 - DNA, 4 bp, artificial sequence
  DpnI restiction enzyme recognition sequence
gatc

SEQ ID NO: 30 - DNA, 4 bp, artificial sequence
  Dam methylase recognition sequence
gatc